(19) United States
(12) Patent Application Publication
(10) Pub. No.: US 2024/0398580 A1
(43) Pub. Date: Dec. 5, 2024

(54) Stabilization Members For Expandable Intervertebral Implants, And Related Systems And Methods

(71) Applicant: Medos International Sarl, Le Locle (CH)

(72) Inventors: Meredith Walsh, Providence, RI (US); Phat Luu, New Bedford, MA (US); Roger Berger, Büren (CH); Connor Engstrom, Hopkinton, MA (US); Didier Gonzalez, Binnigen (CH); Stephane Gully, Rixheim (FR); Markus Hunziker, Aaru (CH); Thomas Martin, Riverside, RI (US); Jonathan Melchor, Providence, RI (US); Sean Saidha, Franklin, MA (US)

(21) Appl. No.: 18/804,161

(22) Filed: Aug. 14, 2024

Related U.S. Application Data
(63) Continuation of application No. 17/683,420, filed on Mar. 1, 2022, now Pat. No. 12,090,064.

(51) Int. Cl. A61F 2/44; A61F 2/30

(52) U.S. Cl. CPC A61F 2/447; A61F 2/30734; A61F 2002/30476; A61F 2002/30482; A61F 2002/30492; A61F 2002/30494; A61F 2002/30495; A61F 2002/30505; A61F 2002/30507; A61F 2002/30736

(57) ABSTRACT

An expandable intervertebral cage includes vertically opposed superior and inferior plates and longitudinally opposed distal and proximal wedges disposed between the plates. The wedges define ramps that engage complimentary ramps of the superior and inferior plates to increase a vertical distance between the plates. An actuator is located between the plates, defines a central, longitudinal axis, and is coupled to the wedges such that, as the actuator rotates about the axis, at least one of the wedges moves longitudinally relative to the other, thereby increasing the vertical separation distance. At least one locking component is insertable within a receptacle at least partially defined by the proximal wedge and is configured to transition from an unlocked configuration, where the actuator is rotatable relative to the proximal wedge, to a locked configuration, where the actuator is rotationally affixed relative to the proximal wedge.

### CROSS REFERENCE TO RELATED APPLICATIONS

This application is a continuation of U.S. application Ser. No. 17/683,420 filed Mar. 1, 2022, the contents of which are hereby incorporated by reference in its entirety.

### TECHNICAL FIELD

The present invention relates to expandable intervertebral implants, and more particularly to structures and features for providing such implants with enhanced stability within an intervertebral space.

### BACKGROUND

Removal of an intervertebral disc is often desired if the disc degenerates. Spinal fusion may be used to treat such a condition and involves replacing a degenerative disc with a device such as a cage or other spacer that restores the height of the disc space and allows bone growth through the device to fuse the adjacent vertebrae. Spinal fusion attempts to restore normal spinal alignment, stabilize the spinal segment for proper fusion, create an optimal fusion environment, and allows for early active mobilization by minimizing damage to spinal vasculature, dura, and neural elements. When spinal fusion meets these objectives, healing quickens and patient function, comfort and mobility improve. Spacer devices that are impacted into the disc space and allow growth of bone from adjacent vertebral bodies through the upper and lower surfaces of the implant are known in the art. Yet there continues to be a need for devices that minimize procedural invasiveness yet stabilize the spinal segment and create an optimum space for spinal fusion.

### SUMMARY

According to an embodiment of the present disclosure, an expandable intervertebral cage includes superior and inferior plates opposite each other along a vertical direction, proximal and distal wedges located between the plates and located opposite each other along a longitudinal direction that is substantially perpendicular to the vertical direction. The distal and proximal wedges each define ramped surfaces configured to engage complimentary ramped surfaces of the plates in a manner increasing a vertical distance between the superior and inferior plates. An actuator is located between the plates and defines a central axis oriented along the longitudinal direction. The actuator is coupled to the distal and proximal wedges such that rotation of the actuator about the central axis moves at least one of the wedges relative to the other wedge along the longitudinal direction in a manner increasing the distance. The cage includes at least one locking component insertable within a receptacle that is at least partially defined by the proximal wedge. The at least one locking component is configured to transition from an unlocked configuration, in which the actuator is rotatable about the central axis relative to the proximal wedge, to a locked configuration, in which the actuator is substantially rotationally affixed relative to the proximal wedge.

According to another embodiment of the present disclosure, an expandable intervertebral cage includes superior and inferior plates opposite each other along a vertical direction, proximal and distal wedges located between the plates and located opposite each other along a longitudinal direction that is substantially perpendicular to the vertical direction. The distal and proximal wedges each define ramped surfaces configured to engage complimentary ramped surfaces of the plates in a manner increasing a vertical distance between the superior and inferior plates. An actuator is located between the plates and defines a central axis oriented along the longitudinal direction. The actuator is coupled to the distal and proximal wedges such that rotation of the actuator about the central axis moves at least one of the wedges relative to the other wedge along the longitudinal direction in a manner increasing the distance. The cage includes at least one locking component that is carried by the proximal wedge and is configured to transition from an unlocked configuration, in which the proximal wedge is longitudinally translatable relative to at least one of the plates, to a locked configuration, in which the proximal wedge is substantially longitudinally affixed relative to the at least one plate.

According to an additional embodiment of the present disclosure, an expandable intervertebral cage includes superior and inferior plates opposite each other along a vertical direction, and a single wedge body that has first and second side surfaces opposite each other along a transverse direction that is substantially perpendicular to the vertical direction. The first and second side surfaces are substantially planar from a proximal end of the wedge body to a distal end of the wedge body. The first and second side surfaces are configured to interface with respective planar surfaces defined along respective interior walls of the superior plate and the inferior plate. The single wedge body defines a plurality of angled rails that extend outwardly from the first and second side surfaces along the transverse direction. The angled rails are oriented at respective oblique angles with respect to a central axis of the cage that is oriented along the longitudinal direction. The plurality of angled rails are configured to ride along a respective plurality of angled guide channels defined along the interior walls of the superior plate and the inferior plate in a manner increasing a distance between the superior and inferior plates along the vertical direction. The cage includes an actuator located between the superior and inferior plates and extending along the central axis. The actuator is coupled to the wedge body such that rotation of the actuator about the central axis moves the wedge body along the longitudinal direction in a manner increasing the distance between the plates.

According to a further embodiment of the present disclosure, an expandable intervertebral cage includes superior and inferior plates opposite each other along a vertical direction, proximal and distal wedges located between the plates and located opposite each other along a longitudinal direction that is substantially perpendicular to the vertical direction. The distal and proximal wedges each define ramped surfaces configured to engage complimentary ramped surfaces of the plates in a manner increasing a vertical distance between the superior and inferior plates. An actuator is located between the plates and defines a central axis oriented along the longitudinal direction. The actuator is coupled to the distal and proximal wedges such that rotation of the actuator about the central axis moves at least one of the wedges relative to the other wedge along the longitudinal direction in a manner increasing the distance. The cage includes at least one locking component that is configured to maintain the increased distance. The locking mechanism comprises a ratchet assembly that includes a first plurality of ratchet teeth defined along a flexible ratchet support, and a second plurality of ratchet teeth, wherein at least one tooth of the first plurality of ratchet teeth is configured to travel against the second plurality of ratchet teeth in sequential interdigitating fashion in a first movement direction as the distance increases. The second plurality of ratchet teeth are configured to prevent movement of the at least one tooth in a second movement direction opposite the first movement direction.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the features of the present application, there is shown in the drawings illustrative embodiments. It should be understood, however, that the application is not limited to the precise arrangements and instrumentalities shown. In the drawings:

FIG. 1 is a plan view of an intervertebral implant positioned between adjacent vertebral bodies, according to an embodiment of the present disclosure;

FIG. 2A is perspective view of an intervertebral implant having locking features for maintaining an expanded height of the implant, according to an embodiment of the present disclosure;

FIG. 2B is a sectional side view of the implant illustrated in FIG. 2A, shown in a collapsed configuration;

FIG. 2C is another sectional side view of the implant illustrated in FIG. 2A, showing the implant in an expanded configuration;

FIG. 2D is an enlarged view of portion of the implant illustrated in FIG. 2C;

FIG. 2E is a perspective view of a locking member illustrated in FIGS. 2B-2D;

FIG. 3A is a perspective view of an intervertebral implant receiving a locking member, according to an embodiment of the present disclosure;

FIG. 3B is a sectional side view of the intervertebral implant illustrated in FIG. 3A, showing the locking member coupled therewith;

FIG. 4A is a sectional side view of a portion of an intervertebral implant, shown in a collapsed configuration, and having a locking member in an unlocked configuration, according to another embodiment of the present disclosure;

FIG. 4B is another sectional side view of the implant illustrated in FIG. 4A, showing the implant in an expanded configuration, and the locking member in a locked configuration;

FIG. 4C is a perspective view of the locking member illustrated in FIGS. 4A-4B;

FIG. 5A is a perspective view of an intervertebral implant having a locking member, according to an embodiment of the present disclosure;

FIG. 5B is a sectional perspective view of the implant illustrated in FIG. 5A, shown with a locking member in a locked configuration;

FIG. 5C is a sectional side view of a portion of implant illustrated in FIG. 5A;

FIG. 5D is a perspective view of select components of a locking mechanism of the implant illustrated in FIG. 5A, shown in a locked configuration;

FIG. 5E is a perspective view of the components illustrated in FIG. 5D, shown in an unlocked configuration;

FIG. 6 is a sectional perspective view of a portion of an intervertebral implant having a locking mechanism according to another embodiment of the present disclosure;

FIG. 7A is a proximal end view of a intervertebral implant, with select components removed, showing a locking mechanism according to another embodiment of the present disclosure;

FIG. 7B is a perspective view of the implant illustrated in FIG. 7A, further showing a tool positioned to unlock the locking mechanism;

FIG. 8 is a sectional perspective view of a proximal end of a intervertebral implant, showing a locking mechanism according to another embodiment of the present disclosure;

FIG. 9A is a plan view of a locking mechanism for an intervertebral implant, according to another embodiment of the present disclosure;

FIG. 9B is a sectional side view of a portion of an intervertebral implant having the locking mechanism illustrated in FIG. 9A;

FIG. 10A is a perspective view of a ratchet locking mechanism for an intervertebral implant, according to another embodiment of the present disclosure;

FIG. 10B is a perspective view of an intervertebral implant employing the ratchet locking mechanism illustrated in FIG. 10A;

FIG. 10C is a top view of the implant illustrated in FIG. 10B;

FIG. 11 is a sectional perspective view of an intervertebral implant having a ratchet locking mechanism according to another embodiment of the present disclosure;

FIG. 12 is a perspective view of an intervertebral implant having a ratchet locking mechanism according to another embodiment of the present disclosure;

FIG. 13 is a perspective view of an intervertebral implant having a ratchet locking mechanism according to another embodiment of the present disclosure;

FIG. 14A is a proximal end view of an intervertebral implant having a cam locking mechanism according to another embodiment of the present disclosure;

FIG. 14B is a proximal end view of an intervertebral implant having another type of cam locking mechanism according to another embodiment of the present disclosure;

FIG. 14C is a sectional side view of a portion of the implant illustrated in FIG. 14B;

FIG. 15 is a sectional top view of a portion of an intervertebral implant having a spring locking mechanism according to another embodiment of the present disclosure;

FIG. 16 is a perspective view of an intervertebral implant having a locking mechanism according to another embodiment of the present disclosure;

FIG. 17 is a sectional perspective view of an intervertebral implant having a drag-type locking mechanism according to another embodiment of the present disclosure;

FIGS. 18A and 18B are plan views illustrating example modes of relative motion between respective components of an intervertebral implant;

FIG. 19A is a perspective view of an intervertebral implant having another locking mechanism according to an embodiment of the present disclosure;

FIG. 19B is a perspective view of select components of the locking mechanism illustrated in FIG. 19A;

FIG. 19C is a perspective sectional view of the implant illustrated in FIG. 19A;

FIG. 20A is a perspective view of an intervertebral implant having another locking mechanism according to an embodiment of the present disclosure;

FIG. 20B is a partial exploded view of select components of the locking mechanism illustrated in FIG. 20A;

FIG. 20C is a perspective sectional view of a portion of the locking mechanism illustrated in FIGS. 20A-20B;

FIG. 21A is a perspective view of an intervertebral implant having another locking mechanism according to an embodiment of the present disclosure;

FIG. 21B is a perspective view of select components of the locking mechanism illustrated in FIG. 21A;

FIG. 21C is a perspective sectional view of a the locking mechanism illustrated in FIGS. 21A-21B;

FIG. 22A is a perspective view of an intervertebral implant having another locking mechanism according to an embodiment of the present disclosure;

FIG. 22B is an enlarged perspective view of a portion of the implant illustrated in FIG. 22A;

FIG. 22C is a sectional side view of a portion of the locking mechanism illustrated in FIGS. 22A-22B;

FIG. 23A is a perspective view of an intervertebral implant having a single wedge body for expanding the implant, shown in a collapsed configuration, according to another embodiment of the present disclosure;

FIG. 23B is an exploded view of the implant illustrated in FIG. 23A;

FIG. 23C is a perspective view of the implant illustrated in FIG. 23A, shown in an expanded configuration; and

FIG. 23D is a sectional side view of the implant shown in FIG. 23C.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the scope of the present disclosure. Also, as used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The terms “approximately”, “about”, and “substantially”, as used herein with respect to dimensions, angles, ratios, and other geometries, takes into account manufacturing tolerances. Further, the terms “approximately”, “about”, and “substantially” can include 10% greater than or less than the stated dimension, ratio, or angle. Further, the terms “approximately”, “about”, and “substantially” can equally apply to the specific value stated.

It should be understood that, although the terms first, second, etc. may be used herein to describe various elements, these elements should not be limited by these terms. These terms are instead used to distinguish one element from another. For example, a first element could be termed a second element, and, similarly, a second element could be termed a first element without departing from the scope of the embodiments disclosed herein.

The embodiments disclosed herein pertain to expandable intervertebral implants, such as fusion cages, that have features that enhance implant stabilization within the intervertebral space. The features disclosed herein enhance the stabilization according to various techniques, which include affixing relative positions between various components of the implants in a manner effectively locking the implants at their desired expanded heights. The stabilization techniques described herein also provide enhanced structural support between various components of the implants, such as by reducing relative motion between select components. Reducing relative motion between components provides numerous advantages, including commensurate rejections in wear and stress on various components, thereby providing an enhanced healing environment for the patient.

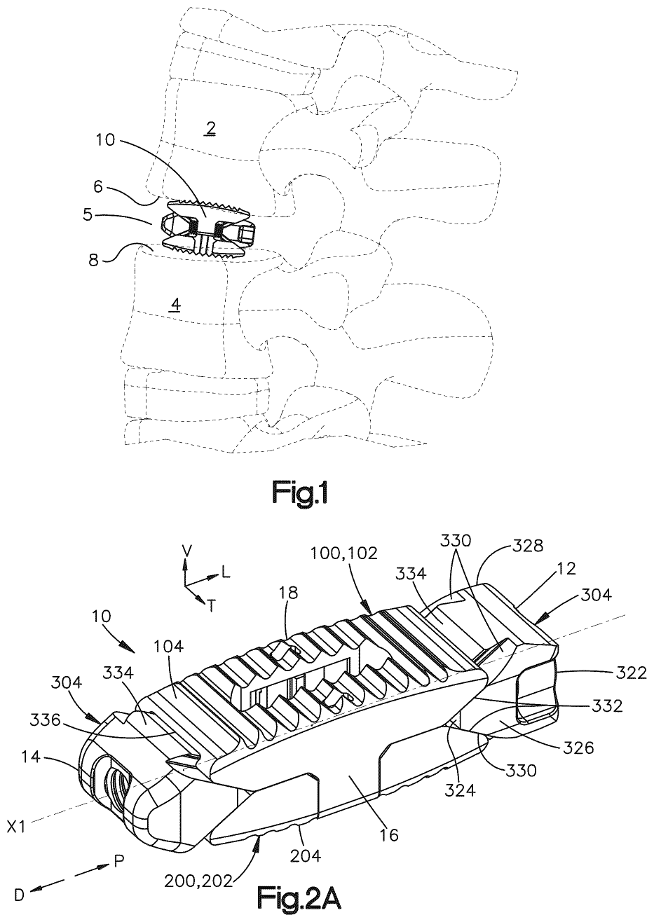

Referring to FIG. 1, a superior vertebral body 2 and an adjacent inferior vertebral body 4 define an intervertebral space 5 extending between the vertebral bodies 2, 4. The superior vertebral body 2 defines superior vertebral surface 6, and the adjacent inferior vertebral body 4 defines an inferior vertebral surface 8. The vertebral bodies 2, 4 can be anatomically adjacent, or can be remaining vertebral bodies after an intermediate vertebral body has been removed from a location between the vertebral bodies 2, 4. The intervertebral space 5 in FIG. 1 is illustrated after a discectomy, whereby the disc material has been removed or at least partially removed to prepare the intervertebral space 5 to receive an expandable intervertebral implant 10. The implant 10 can be configured for use in various regions of the spine, including any of the cervical, lumbar, sacral, and thoracic regions. The implant 10 can also be configured for implantation along various insertion trajectories or “approaches” into the intervertebral space. In the illustrated example, the implant 10 has been inserted along an anterior approach (i.e., from an anterior region of patient anatomy toward a posterior region of the anatomy). In other example embodiments, the implant 10 can be inserted along alternate approaches, such as, by way of non-limiting examples, along a lateral approach, a posterior approach, or a percutaneous approach. It should be appreciated that the implant 10 can also be indicated for various types of intervertebral procedures, including spinal fusions, such as an Anterior Lumbar Interbody Fusion (ALIF), Transforaminal Lumbar Interbody Fusion (TLIF), a Posterior Lumbar Interbody Fusion (PLIF), Lumbar Spinal Fusion Surgery or other surgeries for treating Degenerative Disc Disease (DDD), surgeries for treating Degenerative Spinal Deformity (Def/Deg), minimally invasive surgeries (MIS), and interbody surgeries, by way of non-limiting examples. Once inserted in the intervertebral space 5, the implant 10 can be expanded in a cranial-caudal direction to achieve appropriate height restoration and/or lordosis. The intervertebral space 5 can be disposed anywhere along the spine as desired, including at the lumbar, thoracic, and cervical regions of the spine.

Certain terminology is used in the following description for convenience only and is not limiting. The words “right”, “left”, “lower” and “upper” designate directions in the drawings to which reference is made. The words “inner”, “internal”, and “interior” refer to directions towards the geometric center of the implant, while the words “outer”, “external”, and “exterior” refer to directions away from the geometric center of the implant. The words, “anterior”, “posterior”, “superior,” “inferior,” “medial,” “lateral,” and related words and/or phrases are used to designate various positions and orientations in the human body to which reference is made. When these words are used in relation to the implant 10 or a component thereof, they are to be understood as referring to the relative positions of the implant 10 as implanted in the body as shown in FIG. 1. The terminology includes the above-listed words, derivatives thereof and words of similar import.

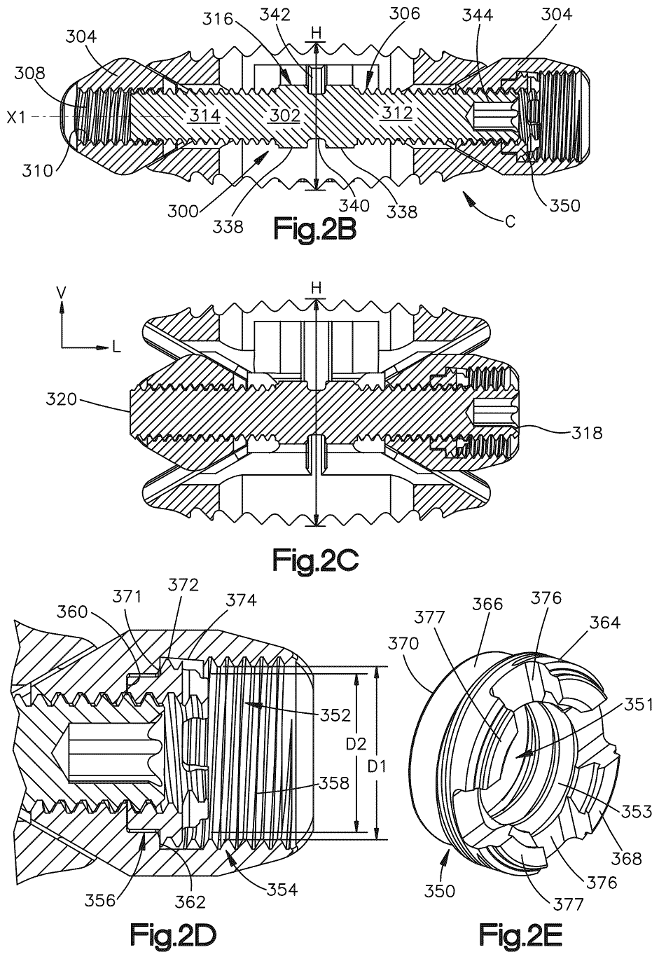

Referring now to FIG. 2A, the implant 10 is described herein as extending horizontally along a longitudinal direction L and a transverse direction T, and vertically along a vertical direction V. Unless otherwise specified herein, the terms “longitudinal,” “transverse,” and “vertical” are used to describe the orthogonal directional components of various implant components and implant component axes. It should be appreciated that while the longitudinal and transverse directions L, T are illustrated as extending along and defining a horizontal plane (also referred to herein as a “longitudinal-transverse plane L-T”), and that the vertical direction is illustrated as extending along a vertical plane (such as either a “vertical-longitudinal plane V-L” or a “vertical-transverse plane V-T,” as respectively referred to herein), the planes that encompass the various directions may differ during use. For instance, when the implant 10 is inserted into the intervertebral space 5, the vertical direction V extends generally along the superior-inferior (or caudal-cranial) direction, while the horizontal plane lies generally in the anatomical plane defined by the anterior-posterior direction and the medial-lateral direction. Accordingly, the directional terms “vertical” and “horizontal” may be used to describe the implant 10 and its components as illustrated merely for the purposes of clarity and illustration.

In FIG. 2A, the implant 10 is shown in a collapsed configuration C. The implant 10 can extend between a proximal or trailing end 12 and a distal or leading end 14 that is spaced from the leading end 14 along a longitudinal implant axis X1 that extends along the longitudinal direction L. The leading and trailing ends 12, 14 may be respectively termed as such because the implant 10 can be inserted leading-end-first into the intervertebral space 5. The trailing end 12 can be configured to couple with one or more insertion instruments, which are configured to support and carry the implant 10 into the intervertebral space 5. The implant 10 can also extend between a first side 16 and an opposed second side 18 along the transverse direction T.

The implant 10 can include a first or superior plate 100 and a second or inferior plate 200 opposing the superior plate 100 along the vertical direction V. The superior plate 100 can define a superior plate body 102 that defines a superior or first bone-contacting surface 104 and the inferior plate 200 can define an inferior plate body 202 that defines an inferior or second bone-contacting surface 204 spaced from the first bone-contacting surface 104 along the vertical direction V. The superior and inferior bone-contacting surfaces 104, 204 can be configured to engage the opposing superior and inferior vertebral bodies 4 and 6, respectively. Each bone-contacting surface 104, 204 can extend in a substantially convex fashion, as shown. The bone-contacting surfaces 104, 204 can be convex along both the longitudinal and transverse directions L, T, although in other embodiments the bone-contacting surfaces 104, 204 can have a convex profile along only one of the longitudinal and transverse direction L, T, and in yet other embodiments the bone-contacting surfaces 104, 204 can be substantially planer. The bone-contacting surfaces 104, 204 can also at least partially define a texture (not shown), such as spikes, ridges, cones, barbs, indentations, or knurls, which are configured to engage the respective vertebral bodies 4 and 6 when the implant 10 is inserted into the intervertebral space 5.

As used herein, the term “distal” and derivatives thereof refer to a direction from the trailing end 12 toward the leading end 14. As used herein, the term “proximal” and derivatives thereof refer to a direction from the leading end 14 toward the trailing end 12. Thus, the implant 10 extends along a proximal direction P from the leading end 14 to the trailing end 12; and the implant 10 also extends along a distal direction D from the trailing end 12 toward the leading end 14. It should be appreciated that the proximal and longitudinal directions P, D are each opposite mono-directional components of the longitudinal direction, which is bi-directional.

As used herein, the term “superior” and derivatives thereof refer to a direction from the second bone-contacting surface 204 toward the first bone-contacting surface 104. As used herein, the term “inferior” and derivatives thereof refer to a direction from the first bone-contacting surface 104 toward the second bone-contacting surface 204. Thus, as used herein, the term “vertical direction V” is bi-directional and is defined by the mono-directional superior and opposed inferior directions.

Referring to FIGS. 2B-2C, the implant 10 can include an expansion mechanism 300 interposed between portions of the superior and inferior plates 100, 200 and configured to separate the superior and inferior plates 100, 200 relative to each other along the vertical direction V. For example, the expansion mechanism 300 can be configured to actuate the implant 10 from the collapsed configuration C, as shown in FIG. 2B, into an expanded configuration E, as shown in FIG. 2C.

The superior and inferior plates 100, 200 can each define features configured to house components of the expansion mechanism 300. For example, the superior and inferior plates 100, 200 can define respective cavities, voids, lumens, spaces, and the like, that are configured to house and/or interface with respective features of the expansion mechanism. Thus, it should be appreciated that portions of the superior and inferior plates 100, 200, such as interior surfaces thereof, that interface with respective features of the expansion mechanism can themselves be characterized as being features of the expansion mechanism. Stated differently, the expansion mechanism can include portions of the superior and inferior plates 100, 200.

The expansion mechanism 300 includes an actuator 302, which can be elongate along the longitudinal direction L. The actuator 302 is configured to push one or more lift bodies 304 of the expansion mechanism 300 against associated portions of one or both of the superior and inferior plates 100, 200 to cause at least one of the superior and inferior plates 100, 200 to move away from the other of the superior and inferior plates 100, 200 along the vertical direction V. In this manner, the actuator 302 is configured to drive vertical expansion of the superior and inferior plates 100, 200, thereby increasing a height H measured between respective portions of the first and second bone-contacting surfaces 104, 204. In particular, the actuator 302 and the one or more lift bodies 304 are configured to increase the height from a first or minimum height H, measured when the implant 10 is in the collapsed configuration C (FIG. 2B), to a second or maximum height H, measured when the implant 10 is in the expanded configuration E (FIG. 2C). It should be appreciated that the expansion mechanism 300 can be configured to controlled expansion to virtually any height between the minimum and maximum heights.

The actuator 302 is configured to move in a manner to push or otherwise force the one or more lift bodies 304 against the associated portions of one or both of the superior and inferior plates 100, 200 to drive expansion of the implant 10 (i.e., to increase the height between the first and second bone-contacting surfaces 104, 204). In the present illustrated example embodiment, the actuator 302 is a shaft configured to rotate about a central axis, which can be the longitudinal implant axis X1. It should be appreciated that axis X1 can also be referred to as the “shaft axis” X1. The shaft 302 has one or more transmission structures 306 configured to transmit rotational movement of the shaft 302 to another mode of movement to the lift bodies 304. In this embodiment, the one or more lift bodies 304 include a pair of wedges 304 engaged with the shaft 302. The wedges 304 define respective channels, which in this example are bores 308, through which the shaft 302 extends. Within the bores 308, interior surfaces of the wedges 304 define complimentary transmission structures 310 configured to engage the transmission structures 306 of the shaft 302. In this example embodiment, the transmission structures 306 of the shaft 302 are exterior threads 306, and the transmission structures 310 of the wedges 304 are interior threads 310 configured to threadedly engage (i.e., intermesh with) the exterior threads 306 of the shaft 302. In this manner, the complimentary exterior threads 306 of the shaft 302 are interior threads 310 of the wedges 304 are configured to transmit rotational movement of the shaft 302 about axis X1 to translational movement of the wedges 304 along the longitudinal direction L. As shown, the exterior threads 306 can be disposed along a first or proximal threaded region 312 and a second or distal threaded region 314 of the shaft 302. The first and second threaded regions 312, 314 can be spaced from each other, such as on opposite sides of a guide formation 316 of the shaft 302.

In the illustrated embodiment, the threads 306 of the first and second threaded regions 312, 314 can extend along opposite thread paths, such that rotating the shaft 302 about axis X1 causes a first or proximal wedge 304 and a second or distal wedge 304 of the pair to translate in opposite directions along the longitudinal direction L. For example, as shown, the proximal and distal wedges 304 can be located at opposite ends 318, 320 of the shaft 302 in the collapsed configuration C and can move toward each other during expansion. However, in other embodiments in which the first and second threaded regions 312, 314 extend along opposite thread paths, the threads 306 can be configured to translate the wedges 304 away from each other during expansion, such as from a central start position adjacent the guide formation 316, and from there toward the opposite ends 318, 320 of the shaft 302. In yet other embodiments, the threads 306 of first and second threaded regions 312, 314 can extend along substantially similar threads paths that cause the wedges 304 to translate in the same direction responsive to rotation of the shaft 302. Moreover, as in the illustrated embodiment, the threads 306 of first and second threaded regions 312, 314 can have a substantially equivalent thread pitch, such that the wedges 304 translate at the same rate responsive to shaft 302 rotation. However, in other embodiments, the threads 306 of first and second threaded regions 312, 314 can have different thread pitches, such that the wedges 304 translate at different rates responsive to shaft 302 rotation.

Referring again to FIG. 2A, each wedge 304 extends from a first or outer end 322 to a second or inner end 324 along the longitudinal direction L. Each wedge 304 also extends from a first side 326 to a second side 328 opposite each other along the transverse direction T. The wedges 304 also have one or more push surfaces 330 that abut one or more complimentary contact surfaces 332 of the superior plate 100 and/or the inferior plate 200. In the illustrated embodiment, each wedge 304 has a superior push surface 330 and an inferior push surface 330 spaced from each other along the vertical direction V. The push surfaces 330 can each be inclined with respect to the vertical direction V. The complimentary contact surfaces 332 of the superior plate 100 and/or the inferior plate 200 are inclined in complimentary fashion with the respective push surface 330 of the wedges 304. The wedges 304 can also include one or more guide formations 334 configured to ride along one or more complimentary guide channels 336 in the superior plate 100 and/or the inferior plate 200 for guiding translational movement of the wedges 304 relative to the superior and inferior plates 100, 200, and further for guiding vertical expansion of the plates 100, 200. For example, in the present embodiment, the wedges 304 can include a superior guide formation 334 and an inferior guide formation 334, which can be superior and inferior protrusions 334 that extend outwardly from the respective push surface 330 along the vertical direction V. The guide formations 334 can ride along complimentary guide channels 336 that extend into the superior and inferior plates 100, 200 from the respective contact surfaces 332. As shown, the guide formations 334 and complimentary guide channels 336 can have intermeshed dovetail geometries.

The superior and inferior plates 100, 200 can define vertical lumens 106, 206 that extend vertically though the plates 100, 200 and have respective openings at the first and second bone-contacting surfaces 104, 204. The lumens 106, 206 can optionally receive bone graft or other bone-growth inducing material, such as after expansion of the implant 10. The guide formation 316 of the shaft 302 can include a flange structure, such as a pair of longitudinally spaced flanges 338 located on opposite sides of an annular recess 340. One or both of the superior and inferior plates 100, 200 can define a guide protrusion 342 that is configured to ride along the annular recess 340 during implant expansion. In this manner, the guide protrusion(s) 342 and the annular recess 340 can further guide vertical expansion of the superior and inferior plates 100, 200 away from each other. The superior and inferior endplates 100, 200 can include additional guide structures. For example, one of the plates 100, 200 can define a vertical extension 346 that is configured to ride along a complimentary vertical channel 348 in the other of the plates 100, 200. Other guide structures are within the scope of the present disclosure.

The shaft 302 defines a drive feature, such as a socket 344, located at the proximal end 318 of the shaft 302. The socket 344 is configured to receive a drive force, such as a rotational drive force, such as a drive torque, from a drive member, such as a drive bit, which can be manually powered or electrically powered. Rotation of the shaft 302 about axis X1 causes the wedges 304 to ride along the first and second threaded region 312, 314 of the shaft 302, which in-turn causes the push surfaces 330 of the wedges 304 to ride along and press against the complimentary contact surfaces 332 of the superior and inferior plates 100, 200, thereby driving the plates 100, 200 away from each along the vertical direction V and thus expanding the implant 10. During expansion, the guide formations 334 also ride along the complimentary guide channels 336 of the superior and inferior plates 100, 200, during which the guide formations 334 can also press against the surfaces of the guide channels 336 in a manner further pressing the plates 100, 200 away from each other. It should be appreciated that the implant 10 and various components thereof can be configured as more fully described in any one of U.S. Pat. No. 8,105,382, issued Jan. 31, 2012, in the name of Olmos et al.; and U.S. Pat. No. 9,717,601, issued Aug. 1, 2017, in the name of Miller, the entire disclosures of which are incorporated by reference herein.

The expansion mechanism 300 is configured such that, after the implant 10 has expanded to the desired height H, the expansion mechanism 300 can be effectively “locked” or otherwise affixed in place to maintain the expanded height H. As shown in FIGS. 2B-2E, the expansion mechanism 300 can include at least one locking member to affix the expansion mechanism 300 in place at the desired height H. In the present embodiment, the at least one locking member is a nut 350, such as a jam nut, that is threaded onto the shaft 302 at the proximal end 318 thereof and configured to abut a portion of the proximal wedge 304 in a manner resisting proximal movement of the proximal wedge 304, thereby securing the relative position of the wedge 304 with respect to the shaft 302, thus locking the expansion mechanism 300 in place, thus affixing the implant at the expanded height H. The jam nut 350 can be disposed along the first threaded region 312 and can extend within a locking receptacle 352 of the proximal wedge 304. The locking receptacle 352 can be in communication with the bore 308 of the proximal wedge 304. The locking receptacle 352 can include a first or proximal receptacle portion 354 and a second or distal receptacle portion 356. The distal receptacle portion 356 can define an inner diameter D2 that is less than an inner diameter D1 of the proximal receptacle portion 354. In the present embodiment, the proximal receptacle portion 354 can define internal threads 358, and the distal receptacle portion 356 can be unthreaded. The inner diameter D1 of the proximal receptacle portion 354 can be defined as the minor thread diameter of the internal threads 358 (i.e., measured at the crests of the internal threads 358). The distal receptacle portion 356 can define a distal end surface 360 of the locking receptacle 352. The distal end surface 360 can provide a distal abutment surface for the jam nut 350. A shoulder surface 362 at a distal end of the proximal receptacle portion 354 can provide an intermediate abutment surface for the jam nut 350.

The jam nut 350 can be configured to threadedly engage the exterior threads 306 of the shaft 302, particularly the proximal threaded region 312 thereof, and to reside in the locking receptacle 352 of the proximal wedge 304. The jam nut 350 can define a central bore 351 and internal threads 353 therein that are configured to threadedly engage the threads of the proximal threaded region 312 of the shaft 302. The jam nut 350 can be configured to reside, in nesting fashion, within the proximal and distal receptacle portions 354, 356. For example, the jam nut 350 can include a proximal nut portion 364 that is configured to reside within with the proximal receptacle portion 354 and can further include a distal nut portion 366 that is configured to reside with the distal receptacle portion 356. The proximal nut portion 364 can define a proximal end 368 of the nut and the distal nut portion 366 can define a distal end 370 of the nut 350. A distal shoulder surface 371 of the proximal nut portion 364 can be configured to abut the interior shoulder surface 362 within the locking receptacle 352. The proximal nut portion 364 can define external threads 372 configured to threadedly engage the internal threads 358 of the proximal receptacle portion 354. The distal nut portion 366 can be smooth and devoid of exterior threads. The proximal nut portion 364 can define an outer diameter that is greater than an outer diameter defined by the distal nut portion 368. The outer diameter of the proximal nut portion 364 can be defined as the major thread diameter of external threads 372.

The jam nut 350 can be “integrated” within the locking receptacle 352 of the proximal wedge 304, meaning that once the jam nut 350 is advanced within the proximal and distal receptacle portions 354, 356 in nesting fashion, the proximal wedge 304, the jam nut 350, and the shaft 302 can be cooperatively configured to retain the jam nut 350 therein. For example, the jam nut 350 can be retained within the locking receptable 352 as a result of the respective thread pitch differences between the threaded interfaces of: (1) the nut external threads 372 with the internal threads 358 of the proximal wedge 304; and (2) the nut internal threads 353 with the threads of the proximal threaded region 312 of the shaft 302. It should be appreciated that the proximal wedge 304 can employ other types of retention features for retaining the jam nut 350 therein.

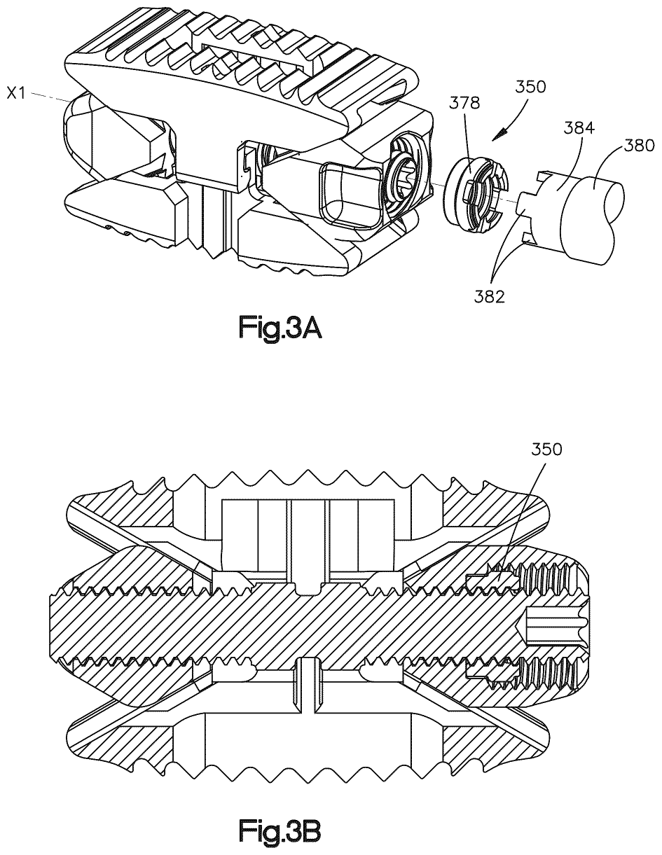

The proximal nut portion 364 can define one or more mounting formations for coupling with a tool, such as a nut insertion tool, such as a tool similar to the nut insertion tool 380 described below with reference to FIG. 3A. For example, with continued reference to FIGS. 2B-2D, the proximal nut portion 364 can define a plurality of receptacles 376, which can be circumferentially spaced between associated mounting protrusions 377 that extend in the proximal direction. As shown, the receptacles 376 can be arranged in a cross-pattern, which can be configured to receive complimentary mounting teeth 382 defined at a distal end 384 the tool 380. The complimentary geometries of the receptacles 376 and teeth 382 can be configured such that the jam nut 350 is secured to the distal end 384 of the tool 380 while the tool 380 inserts the jam nut 350 with the locking receptacle 352. The complimentary geometries of the receptacles 376 and teeth 382 are further configured such that the tool 380 can rotate the jam nut 350 about a central tool axis colinear with the shaft axis X1 to threadedly engage the external threads 372 of the nut 350 along the internal threads 358 of the proximal receptacle portion 354 until the external threads 372 have distally cleared the internal threads 358, at which time the interior retention surface(s) 374 retain the jam nut 350 within the proximal receptacle portion 354.

The mounting protrusions 377, or at least a portion thereof, can optionally possess a degree of flexibility so that, for example, once the external threads 372 have distally cleared the internal threads 358, such flexible portions of the mounting protrusions 377 can deflect radially outward, further retaining the jam nut 350 within the locking receptacle 352. Moreover, such flexible portions of the mounting protrusions 377 can produce audible and/or tactile feedback, such as a clicking sensation, as they deflect radially outward, thereby alerting the technician once the jam nut 350 is retained within the locking receptacle 352.

After the jam nut 350 is retained with the locking receptacle 352, the tool 380 can be withdrawn by simply proximally translating the tool 380. After the jam nut 350 is retained within the proximal receptacle portion 354, the interior retention surfaces 374 cause the jam nut 350 to threadedly travel along the proximal threaded region 312 of the shaft 302 concurrently with the proximal wedge 304 responsive to rotation of the shaft 302 about the shaft axis X1. In this manner, once the implant 10 has expanded to the desired height H, the jam nut 350 can remain in abutting contact against the proximal wedge 304, such as at one or both of the distal end surface 360 and shoulder surface 362 within the locking receptacle 352, thereby preventing the proximal wedge 304 from backing out along the shaft 302, and thus affixing the implant 10 at the desired height H.

Referring now to FIGS. 3A-3B, in other embodiments, the jam nut 350 can be detached or “non-integrated.” In such embodiments, the proximal nut portion 364 of the jam nut 350 can define an outer surface 378 that is devoid of external threads. The outer surface 378 can be substantially smooth and can define the outer diameter of the proximal nut portion 364. It should be appreciated that the jam nut 350 of the present embodiment can otherwise be substantially similar to the jam nut 350 described above with reference to FIGS. 2B-2D. In the present embodiment, the outer diameter of the proximal nut portion 364 can be less than the inner diameter D1 of the proximal receptacle portion 354 (which, as above, is the minor diameter of the internal threads 358). In such embodiments, the insertion tool 380 can insert the jam nut 350 into the locking receptacle 352 after the shaft 302 has been rotated to expand the implant 10 to the desired height H. In particular, the insertion tool 380 can advance the jam nut 350 within the locking receptacle 352, such as by rotating the nut 350 into threaded engagement with the proximal threaded region 312 of the shaft until the nut 350 distally abuts the proximal wedge 304, such as at one or both of the distal end surface 360 and shoulder surface 362 within the locking receptacle 352. Once abutting contact has been made, the tool 380 can be proximally withdrawn. Moreover, as described above, once the jam nut 350 distally abuts the proximal wedge 304, the jam nut 350 impedes the proximal wedge 304 from threadedly backing out along the shaft 302, thereby affixing the implant 10 at the desired height H1.

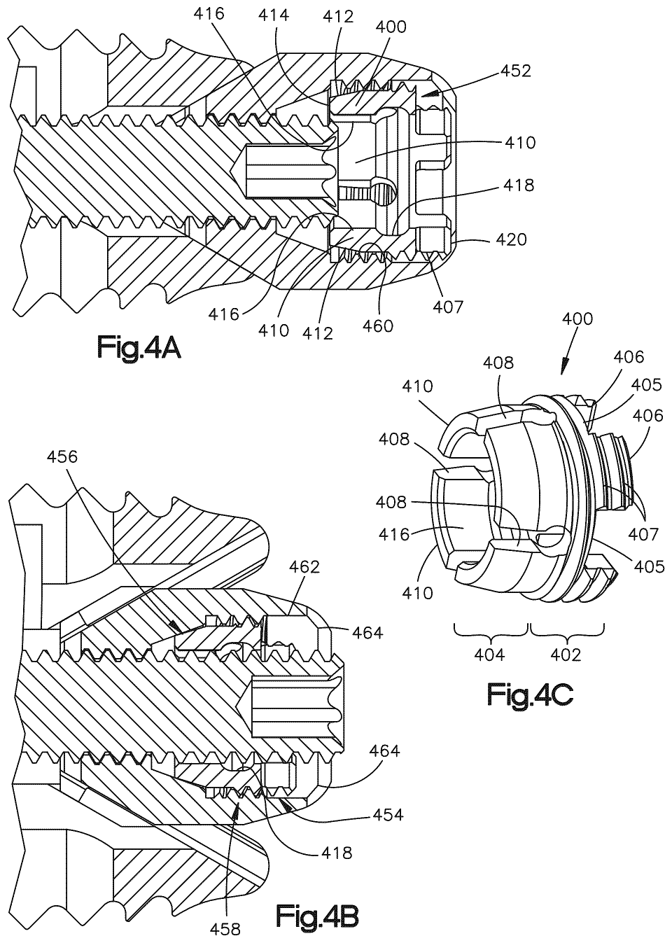

Referring now to FIGS. 4A-4B, in other embodiments, the at least one locking member can be a collet 400 configured for insertion with the locking receptacle 352. The collet 400 defines a proximal portion 402 and a distal portion 404. The proximal portion 402 defines one or more mounting formations, such as recesses 405 and associated mounting protrusions 406 circumferentially spaced from each other in alternating fashion. The recesses 405 and mounting protrusions 406 can be configured for coupling with a tool, such as a tool generally similar to the tool 380 described above. The collet 400 can also define external threads 407, such as along the proximal portion 402. The distal portion 404 of the collet 400 can define a plurality of gaps or cutouts 408 spaced about the circumference of the collet 400 so as to define a plurality of extensions or legs 410 circumferentially spaced between the gaps 408. Outer surfaces 412 of the legs 410 preferably taper radially inwardly toward the distal direction, such that the outer diameter of the distal collet portion 404 diminishes toward a distal end 414 of the collet 400. The legs 410 can define inner clamp surfaces 416 adjacent the distal end 414 of the collet 400, which inner clamp surfaces 416 can be configured to press radially inward against the shaft 302, as described in more detail below. The inner clamp surfaces 416 can define an inner diameter that is substantially equivalent to, but slightly greater than, a major thread diameter of the proximal threaded region 312, at least when the collet 400 is in a neutral configuration.

The collet 400 can also define an inner relief surface 418, which can be located between the distal end 414 and a proximal end 420 of the collet 400. The relief surface 418 preferably has an inner diameter that is greater than the inner diameter of the clamp surfaces 416. The collet 400 can also define an inner guide surface adjacent the proximal end 420. When the collet 400 is in the neutral configuration, the inner clamp surfaces 416 can translate across and along the crests of the exterior threads 306 of the proximal threaded region 312.

The proximal wedge 304 can define a locking receptacle 452 that is generally similar to the locking receptacle 352 described above. In the present embodiment, however, the locking receptacle 452 can include proximal and distal receptacle portions 454, 456 and an intermediate receptacle portion 458 located longitudinally between the proximal and distal receptacle portions 454, 456. Within the distal receptacle portion 456, an interior surface 402 of the proximal wedge 304 tapers radially inwardly toward the distal direction in complimentary fashion with the tapered outer surfaces 412 of the legs 410. Thus, an inner diameter of the interior surface diminishes along the distal direction. The proximal wedge 304 can define internal threading 460 within the locking receptacle 452, such as along the intermediate receptacle portion 458 thereof. Within the proximal receptacle portion 454, the wedge 304 can define an interior surface 462 that is substantially devoid of internal threads.

The collet 400 is insertable within the locking receptacle 452 and is configured to distally advance therein from the neutral configuration to a locked configuration. For example, FIG. 4A shows the collet 400 in the neutral configuration, in which the external threads 407 of the collet 400 substantially reside within proximal receptacle portion 454 and are not engaged with the internal threading 460 within the locking receptacle 452. The collet 400 can reside within the locking receptacle 452 in the neutral configuration while the shaft 302 is rotated to advance the wedges 304 and thereby expand the implant 10. The collet 400 can be configured such that, while in the neutral configuration, the shaft 302 can freely rotate relative to the collet 400 about a first rotational direction that expands the implant 10, such as a clockwise direction. After the implant 10 is expanded to the desired height H, the collet 400 can be distally advanced within the distal receptacle portion 456 to transition from the neutral configuration to the locked configuration with the shaft 302. During this transition, the collet 400 can be rotated via the tool such that the external threads 407 of the collet 400 threadedly engage and advance along the internal threading 460 within the locking receptacle 452, which also causes the collet 400 to advance distally with respect to the shaft 302. Distally advancing the collet 400 in this manner advances the collet legs 410 into the distal receptacle portion 454.

The collet legs 410 are preferably configured such that, as the collet 400 advances distally within the locking receptacle 452 and distally relative to the shaft 302, the tapered outer surfaces 412 of the legs 410 contact the tapered interior surface 402 of the distal receptacle portion 456. As the collet 400 further advances distally, the tapered interior surface 402 pushes the legs 410 radially inward, thereby causing the inner clamp surfaces 416 to clamp against the shaft 302 with sufficient clamping force to effectively lock the collet 400 to the shaft 302, and to further lock the collet 400 to the proximal wedge 304. In this manner, the collet 400 can effectively lock the proximal wedge 304 to the shaft 302, thereby affixing the implant 10 at the desired height H1. The clamping force can cause plastic deformation of the shaft exterior threads 306 and/or the inner clamp surfaces 416 of the legs 410, thereby creating a locking press-fit or crush-fit between the collet legs 410 and the shaft 302. The clamping force can also cause the proximal portion 402 of the collet 400 to flex outward, which can press the collet external threads 407 radially outward against the internal threads 460, thereby increasing the locking engagement between the collet 400 and the proximal wedge 304.

The collet 400 can be integrated within the locking receptacle 452, meaning that the collet 400 and the locking receptacle 452 can be cooperatively configured to retain the collet 400 therein. For example, a proximal end portion of the locking receptacle 452 can include a retention feature, such as one or more circumferential lips 464 that extend radially inward from the interior surface 462. The one or more circumferential lips 464 can have a respective proximal side having an interior thread-like profile, such that the collet external threads 407 can threadedly engage and subsequently clear the lips 464 as the collet 400 is inserted distally into the locking receptacle 452. However, a distal side of the one or more circumferential lips 464 can have a geometry configured to prevent the collet 400 from backing out of the locking receptacle 452. It should be appreciated that in other embodiments the collet 400 can be a separate, detached component that is remote from the implant 10 during expansion and is subsequently insertable within the locking receptacle 452 and into the locked configuration after the implant 10 has expanded to the desired height H.

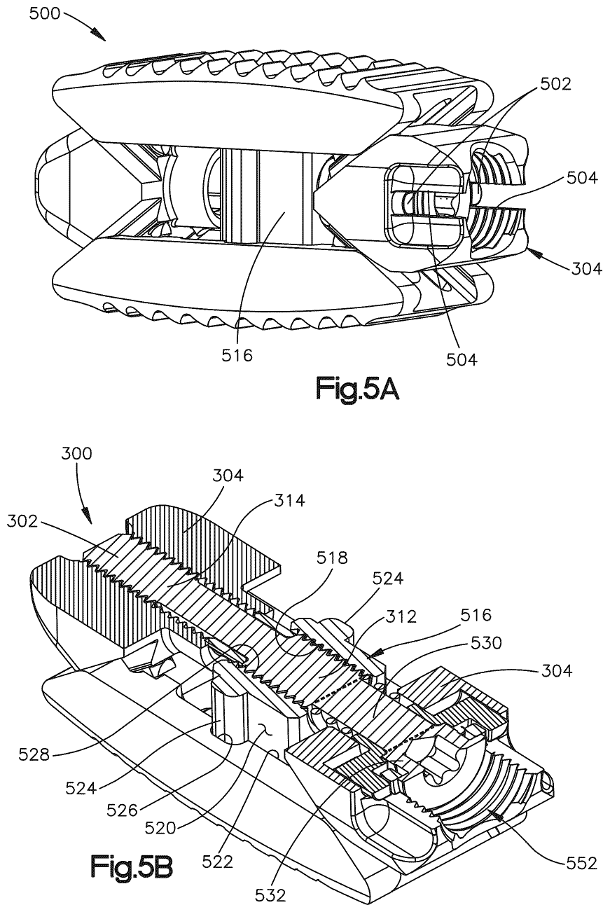

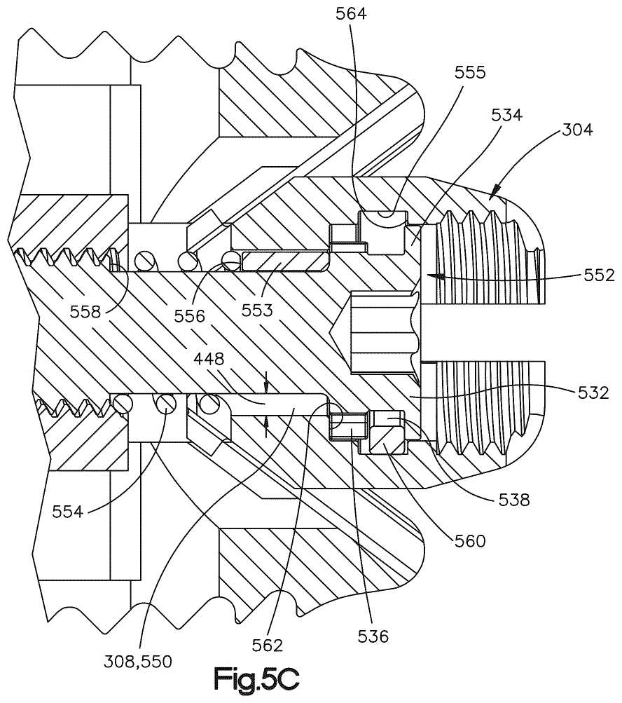

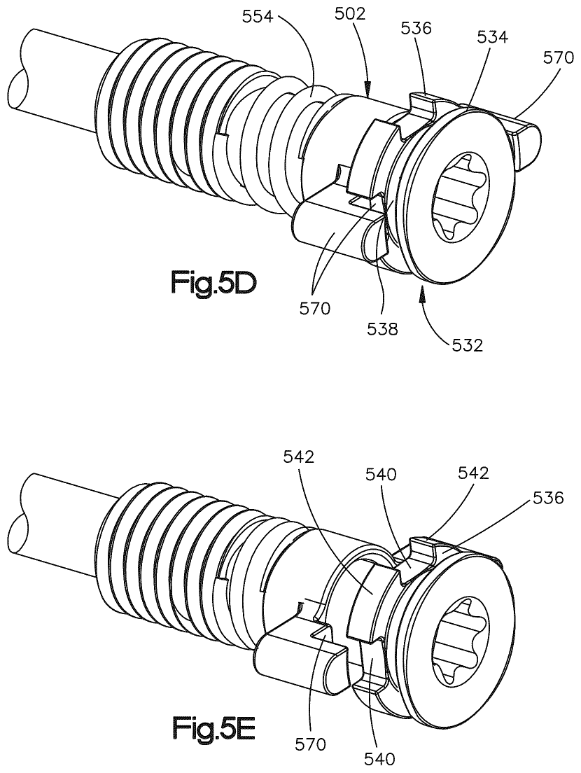

Referring now to FIGS. 5A-5E, an example embodiment of an expandable implant 500 is shown in which the at least one locking member includes a biased locking member, such as a spring-loaded locking plunger 502. The implant 500 can be generally similar to the implants 10 described above. For the sake of brevity, the following disclosure will focus primarily on differences of the implant 500 with respect to the implants 10 described above. The locking plunger 502 can be accessible and actuatable through access slots 504 defined by the proximal wedge 304.

The expansion mechanism 300 of the present embodiment can include a guide hub 516 located longitudinally between the wedges 304. The guide hub 516 can be a component that is separate from the shaft 302 and the superior and inferior plates 100, 200, although in other embodiments the guide hub 516 can be a monolithic extension of the one of the superior and inferior plates 100, 200. The guide hub 516 can define a bore 518 through which the shaft 302 extends. The guide hub 516 can define one or more guide structures for guiding expansion of the implant 500 along the vertical direction V. One such guide structure can be the general shape of the guide hub 516, which can be generally rectangular and/or block-like and can have side surfaces 520 opposite each other along the transverse direction T. The side surfaces 520 of the guide hub 516 can translate along complimentary inner surfaces 522 of the superior plate 100 and/or the inferior plate 200. The guide hub 516 can also define one or more additional vertically oriented guide structures, such as a pair of rails 524 that protrude outwardly from the side surfaces 520 and are received within complimentary guide channels 526 defined in the superior and inferior plates 100, 200. The complimentary rails 524 and guide channels 526 can guide expansion of the superior and inferior plates 100, 200 along the vertical direction V and can also maintain a relative longitudinal position between the guide hub 516 and the plates 100, 200 during expansion. It should be appreciated that other guide structures are within the scope of the present disclosure.

Similar to the embodiments described above, the distal wedge 304 can define interior threads 310 that threadedly engage the distal threaded region 314 of the shaft 302. However, as shown in FIG. 5B, in the present embodiment, the proximal threaded region 312 of the shaft 302 can threadedly engage interior threads 528 defined within the bore 518 of the guide hub 516. The proximal wedge 304 can be coupled to a non-threaded proximal region 530 of the shaft 302 that extends longitudinally between a proximal head 532 of the shaft 302 and the proximal threaded region 312. The head 532 of the shaft 302 can define various mounting formations for coupling the shaft 302 to the proximal wedge 304 and the locking plunger 502. For example, with reference to FIGS. 5B-5E, the head 532 can include a first flange 534, a second flange 536 distally spaced from the first flange 534, and an annular receptacle 538 located longitudinally between the first and second flanges 534, 536. The head 532 can be configured to reside within a locking receptacle 552 defined within the bore 308 of the proximal wedge 304. A retention member, such as a retention clip 560, can reside within the annular receptacle 538 of the head 532 (i.e., between the flanges 534, 536) and can also reside in an annular receptacle portion 555 of the locking receptacle 552 of the proximal wedge 304. The head 532 and the retention clip 560 can have complimentary geometry with the locking receptacle 552 of the wedge 304 so as to maintain a relative longitudinal position between the proximal wedge 304 and the head 532 (and thus also between the proximal wedge 304 and the shaft 302). For example, as best shown in FIG. 5C, the second flange 536 of the head 532 can abut a distal shoulder surface 562 of the wedge 304 within the locking receptacle 552. The retention clip 560 can fit snugly between the flanges 534, 536 of the head 532 and can also be configured to abut a proximal shoulder surface 564 at a proximal end of the annular receptacle portion 555. Thus, the second flange 536 can substantially prevent the proximal wedge 304 from translating proximally relative to the shaft 302, and the retention clip 560 can substantially prevent the proximal wedge 304 from translating distally relative to the shaft 302. In this manner, rotation of the shaft 302 of the present embodiment can cause the distal wedge 304 to translate proximally along the distal threaded region 314, while the proximal threaded region 312 travels distally along the interior threads 528 within the bore 518 of the guide hub 516, thereby also causing the proximal wedge 304 to travel distally along with the shaft 302 relative to the guide hub 516 and the superior and inferior plates 100, 200, thereby also expanding the implant 10 along the vertical direction V.

As best shown in FIGS. 5B-5C, the locking plunger 502 can also be configured to reside within the locking receptacle 552 and is configured to engage the head 532 in locking fashion, as described in more detail below. A distal portion 550 of the bore 308 of the proximal wedge 304 can be sized to provide an annular gap 448 between the distal bore portion 550 and the non-threaded proximal region 530 of the shaft 302. A distal extension 553 of the locking plunger 502 can extend within the gap 448 and can engage a bias member 554, which can be configured to bias the locking plunger 502 into locked engagement with the head 532. The bias member 554 can be a spring, such as a compression coil spring 554 that extends between a first push surface 556 defined at a distal end of the distal extension 553 and an opposed second push surface 558, which can be defined at a proximal end of the proximal threaded region 312 of the shaft 302.

Referring now to FIGS. 5D-5E, the second flange 536 can define a plurality of recesses 540 spaced about the circumference of the second flange 536. The recesses 540 effectively provide the second flange 536 with a plurality of protrusions or teeth 542 extending respectively between adjacent ones of the recesses 540 about the circumference of the second flange 536. The spring 554 is configured to bias the locking plunger 502 proximally so that locking arms 570 of the locking plunger 502 advance within the recesses 540 when the recesses 540 are rotationally aligned with the locking arms 570. When the locking arms 570 reside in the recesses 540, the shaft 302 is substantially prevented from rotating, thereby affixing the longitudinal positions of the wedges 304 with respect to the shaft 302, thereby also affixing the implant 500 at the associated height H. Thus, when the locking arms 570 reside in the recesses 540, the locking plunger 502 is in a locked position. To disengage the locking arms 570 from the recesses 540 and thereby move the plunger 502 to an unlocked position, an unlocking tool having one or more detent features, such as push arms, can be configured to extend within the access slots 504 of the proximal wedge 304 and press the locking arms 570 distally with sufficient force to overcome the bias force imparted by the spring 554, thereby pushing the locking arms 570 out of engagement with the recesses 540, at which position the shaft 302 can be rotated to drive expansion of the implant 500.

It should be appreciated that the unlocking tool can be part of a multi-component instrument for expanding the implant 500. For example, the unlocking tool can have an elongate tubular body that includes the push arms at a distal end thereof. The tubular body can define a lumen, through which a drive tool of the instrument can be advanced for coupling with the shaft 302, such as within the socket 344. At such position, and with the unlocking tool pressing the locking plunger 502 into the unlocked position, the drive tool can be driven to rotate the shaft 302 and expand the implant 500 to the desired height H. Once the implant 500 expands to or near the desired height, the unlocking tool can be withdrawn a distance, removing the counter force against the spring 554 bias force. If the recesses 540 of the second flange 536 are rotationally aligned with the locking arms 570, the locking arms 570 can extend into the recesses 540 and thus into the locked position. If the recesses 540 are not rotationally aligned with the locking arms 570 when the unlocking tool is withdrawn, the drive tool can be used to selectively rotate the shaft clockwise or counterclockwise a measure (thus also slightly expanding or contracting the height H) until the recesses 540 are aligned with the locking arms 570, at which time the locking arms 570 will be biased into the recesses 540 and into the locked position.

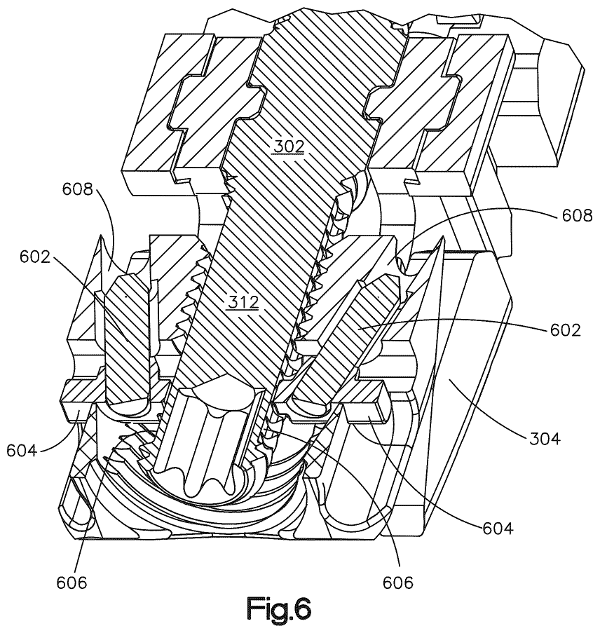

It should be appreciated that the embodiments described above represent non-limiting examples of locking features for affixing the implant at the desired height H. Additional non-limiting examples are described below with reference to FIGS. 6-17.

Referring now to FIG. 6, in additional embodiments, the at least one locking member can include a pair of pins 602 coupled to respective sliders 604 that extend within opposed locking channels 606 that extend longitudinally along the proximal threaded portion 312 of the shaft 302. The pins 602 and sliders 604 are shown in a locked position with the shaft 302, in which position the sliders 604 reside within the locking channels 606 and inhibit rotation of the shaft 302. The pins 602 extend within guide slots 608 defined within the proximal wedge 304. The guide slots 608 are angled outwardly from the shaft 302 so that distal advancement of the sliders 604 guides the pins 602 and sliders 604 out of the locking channels 606, thereby allowing the shaft 302 to rotate.

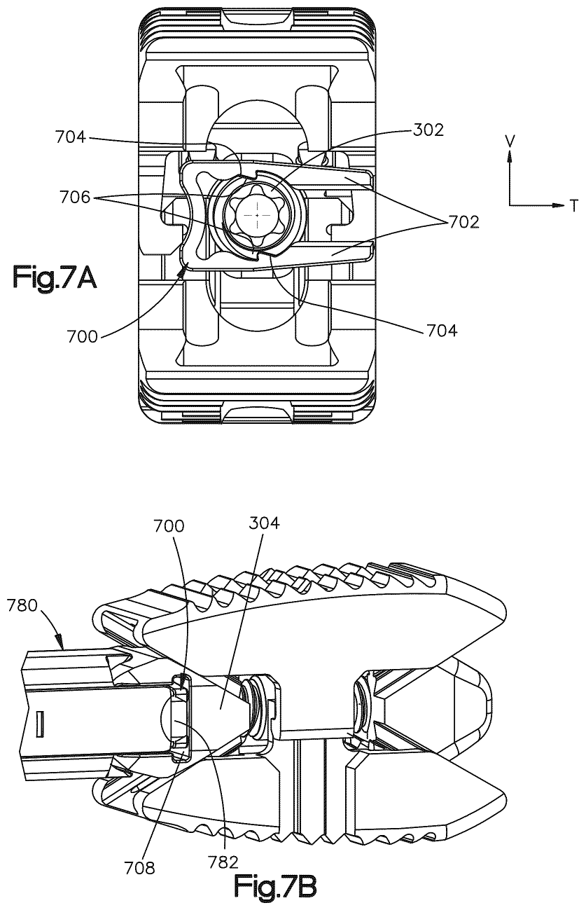

Referring now to FIGS. 7A-7B, the at least one locking member can be a flexible member, such as a clip spring 700, which can define one or more retention formations 704 for gripping the shaft 302 in a manner preventing shaft rotation about axis X1. The clip spring 700 can include a pair of spring arms 702 that define the retention formations 704 and bias them against the shaft 302 along a direction substantially perpendicular to axis X1, such as along the vertical direction V, for example. The retention formations 704 can be biased against complimentary surfaces, such as serrations 706, defined by the shaft 302. As shown in FIG. 7B, the clip spring 700 can reside within a receptacle 708 defined by the proximal wedge 304. The clip spring 700 can be unlocked by driving the spring arms away from each other, such as via a tool 780 having a protrusion 782 configured to extend between the spring arms 702 and force the arms 702 apart as the tool 780 advances distally. With the spring arms 702 moved away from each other, the retention formations 704 decoupled from the serrations 706, allowing the shaft 302 to rotate.

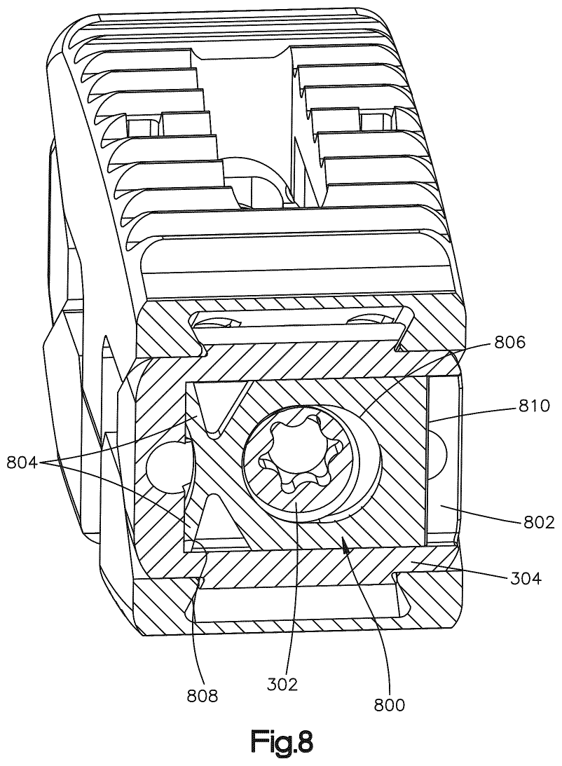

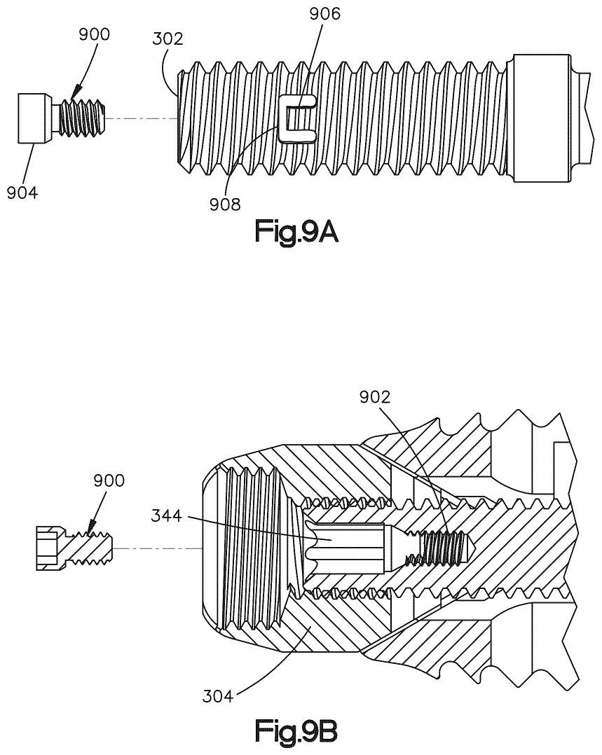

Referring now to FIG. 8, in additional embodiments, the locking member can be a button 800, such as a push or slide button. As shown, the button 800 can reside in a transverse receptacle 802 defined within the proximal wedge 304. The button 800 can define a bore 806, which can be eccentric, and through which the shaft 302 extends. The button 800 can define spring arms 804 that impinge against a wall 808 of the receptacle 802 and bias the button 800 against the shaft 302 in a manner inhibiting shaft rotation. To move the button 800 into an unlocked position, a tool can engage an end 810 of the button 800 opposite the spring arms 804 to slide the button 800 transversely into an unlocked position.

Referring now to FIGS. 9A-9B, the at least one locking member can be insertable within a receptacle of the shaft 302 for deforming a portion of the shaft 302 radially outward against a respective portion of the proximal wedge 304, thereby inhibiting rotation of the shaft 302. For example, such a locking member can be a threaded pin 900, which can be inserted within a threaded receptacle 902, such as at a distal end of the socket 344. As the pin 900 threads within the receptacle 902, a head 904 of the pin 900 can press a flex tab 906 of the shaft 302 radially outward. As shown, the flex tab 906 can be defined by a U-shaped cutout 908 in a body of the shaft 302.

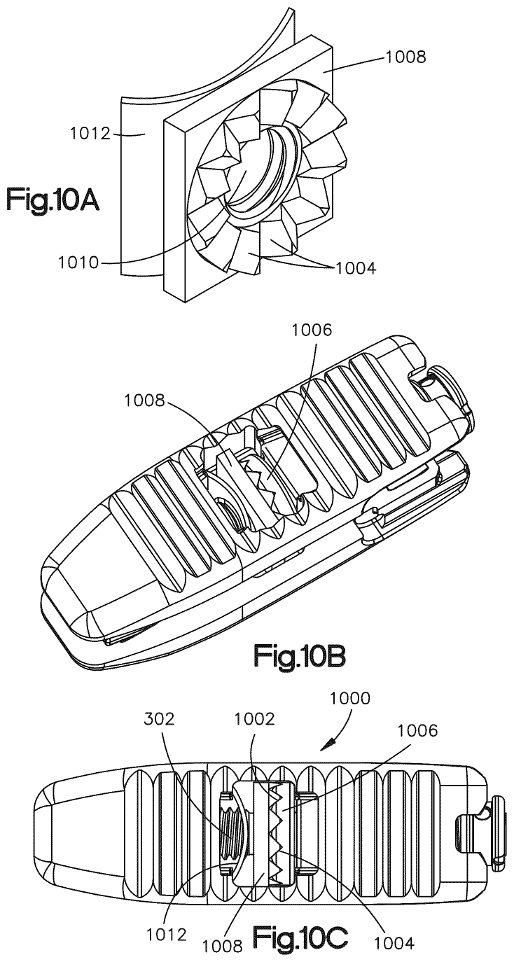

In additional embodiments, the at least one locking member can comprise a ratchet locking assembly, which can have various configurations. Non-limiting examples of such ratchet assemblies will now be described with reference to FIGS. 10A-13.

Referring now to FIGS. 10A-10C, a locking mechanism can comprise a ratchet locking assembly 1000 that includes a first plurality of ratchet teeth 1002 that engage a second plurality of ratchet teeth 1004 as the implant expands. It should be appreciated that each respective plurality of ratchet teeth can also be referred to as a “set” of ratchet teeth. The first set of ratchet teeth 1002 can be defined along a component that travels in close proximity to another component as the implant 10 expands. For example, the first set of ratchet teeth 1002 can be arranged in circular fashion along a flange 1006 of the shaft 302 and can be configured to rotate about the central axis X1 along with the shaft 302. The second set of ratchet teeth 1004 can be arranged in a complimentary circular pattern along a flexible ratchet support 1008, which can bias the first and second pluralities of ratchet teeth 1002, 1004 into engagement with each other. The flexible ratchet support 1008 can also be configured to provide a measure of positional compliance between the first and second pluralities of ratchet teeth 1002, 1004, so that respective leading surfaces of the ratchet teeth 1002, 1004 can slidingly engage each other, such as in sequential interdigitating fashion.

As shown, the flexible ratchet support 1008 can include a support block 1008 that resides in a receptacle between the superior and inferior plates 100, 200. The support block 1008 can define a bore 1010 through which the shaft 302 extends. A spring member, such as a leaf spring 1012, can bias the support block 1008 toward the flange 1006 so that the first and second sets of ratchet teeth 1002, 1004 engage each other. It should be appreciated that one of the first and second sets of ratchet teeth 1002, 1004 need only include a single ratchet tooth for the ratchet assembly 1000 to provide ratcheting locking function for the implant 10. For example, the ratchet assembly 1000 can be configured such that at least one tooth of one set 1002, 1004 travels against at least one tooth of the other set 1004, 1002, and optionally against a plurality of teeth of the other set 1004, 1002, such as in sequential interdigitating fashion along a first movement direction as the implant 10 expands. In some embodiments, it can be said that one of the sets of ratchet teeth 1002, 1004 is configured to prevent movement of at least one tooth of the other set in a second movement direction opposite the first movement direction.

It should be appreciated that, in some embodiments, the sets of ratchet teeth 1002, 1004 need not possess full locking geometries with respect to the second movement direction. In such embodiments, the sets of ratchet teeth 1002, 1004 can merely provide enhanced resistance or “drag” in the second movement direction, which can be sufficient to prevent the implant 10 from losing height in some surgical applications.

Referring now to FIG. 11, in another embodiment, a ratchet locking assembly 1100 includes a first set of ratchet teeth 1102 can be arranged along one or both of the sides 326, 328 of the proximal wedge 304 and/or the distal wedge 304. As shown, the first set of ratchet teeth 1102 can be arranged along both sides 326, 328 of the proximal wedge 304. A second set of ratchet teeth 1104 can be disposed on one or more flexible support arms 1108, such as a pair of flexible support arms 1108, which can extend longitudinally from a block member 1110, such as a bearing member, vertically disposed between the superior and inferior plates 100, 200.

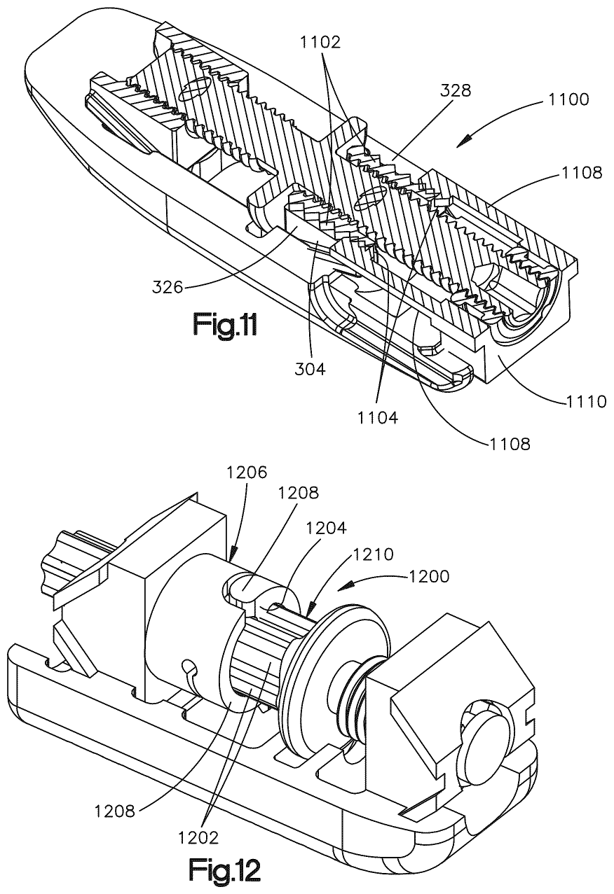

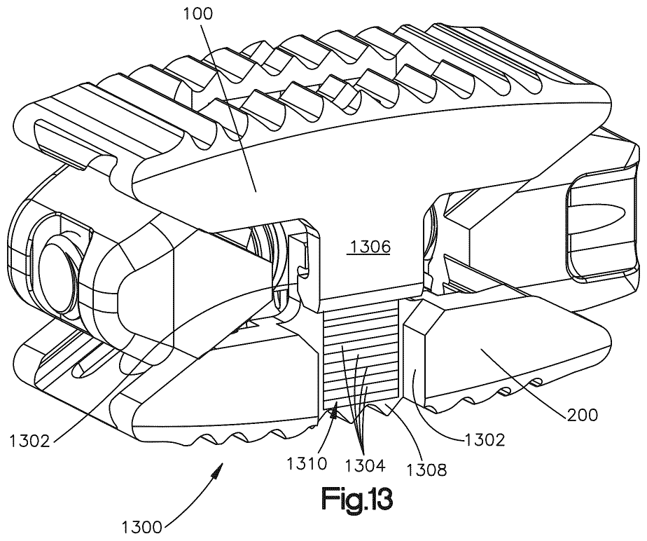

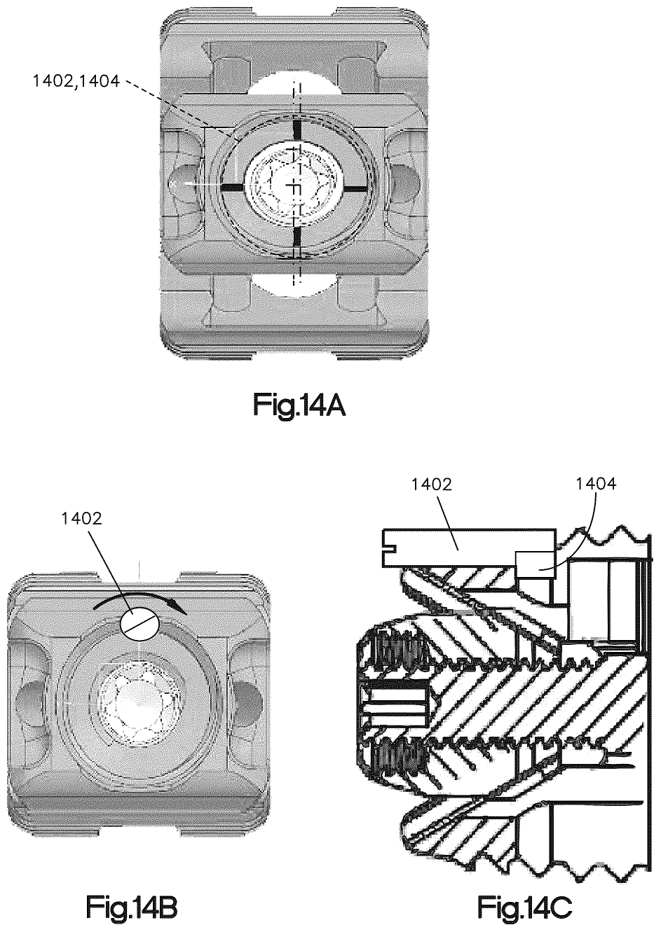

Referring now to FIG. 12, a ratchet locking mechanism 1200 can include flexible ring member, such as a ring clip 1206 having flexible spring arms 1208 configured to ride along ratchet teeth 1202 in the form of radial protrusions defined along an outer surface of a shaft collar 1210 as the collar 1210 rotates with the shaft 302. Respective inner protrusions 1204 at the ends of the spring arms 1208 can function as pawls that allow the collar 1210 (and thus the shaft 302) to rotate about a first rotational direction to expand the implant 10, but inhibit or at least resist shaft rotation about an opposite rotational direction.

Referring now to FIG. 13, at least one first ratchet tooth 1302 can be defined on an inner surface of a vertical guide extension 1306 of one of the plates 100, 200 (such as the superior plate 100), and the at least one first ratchet tooth 1302 can be configured to engage a second set of ratchet teeth 1304 that are defined along an outer surface 1308 within a vertical guide channel 1310 defined by the other one of the plates 200, 100 (such as the inferior plate 200).

Referring now to FIG. 14A-14C, in some embodiments, the at least one locking member can comprises a cam member 1402 that defines a cam surface 1404 and is rotatable about an axis, during which the cam surface can move from a disengaged position to an engaged position. When in the engaged position, the cam surface 1404 can push against the shaft 302 or against one of the wedge 304, such as the proximal wedge 304, in a manner locking the relative positions between the shaft 302 and the wedges 304. As indicated by the dashed outline in FIG. 14A, the cam member 1402 can be an eccentric ring and/or washer, which can have an inner surface configured to press against the shaft 302 as the ring/washer rotates to the locked position.

Alternatively, as shown in FIGS. 14B-14C, the cam member 1402 can be a pin, which can reside within a receptacle defined within one of the superior and inferior plates 100, 200 (such as the superior plate 100). The pin 1402 can be accessible after the implant 10 has expanded to a certain height, for example. A distal end of the pin 1402 can define an eccentric nose 1404 that defines the cam surface. Once the implant has expanded sufficiently to expose or otherwise provide access to the pin 1402, the pin 1402 can be rotated to bring the eccentric nose 1404 into engagement with a complimentary contact surface in a manner inhibiting reduction of the expanded height H. It should be appreciated that various other cam lock structures are within the scope of the embodiments herein.

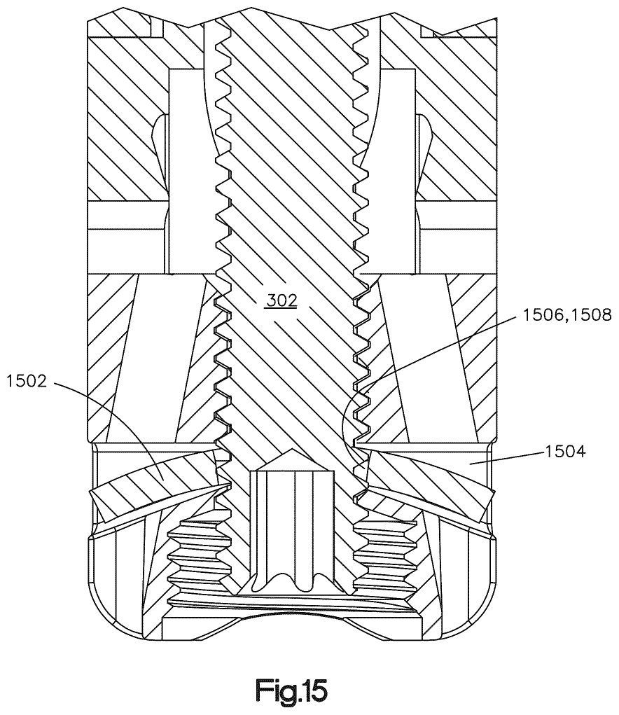

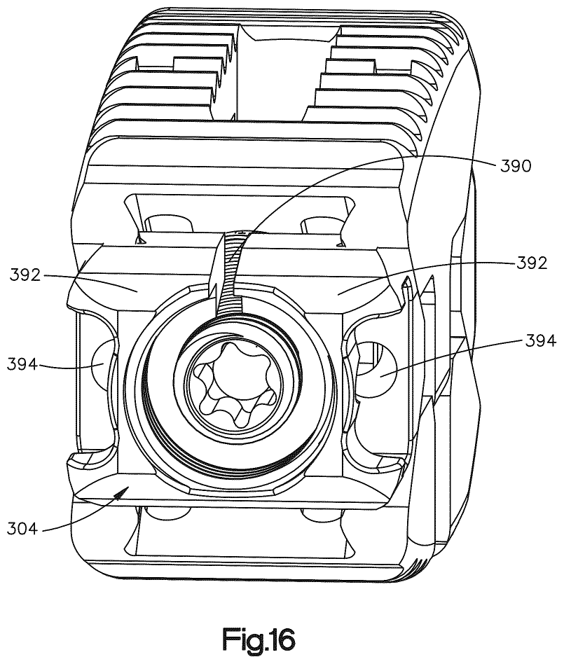

Referring now to FIG. 15, in additional embodiments, the at least one locking member can be a spring member, such as a leaf spring 1502, which can reside within a receptacle 1504 within the proximal wedge 304. The leaf spring 1502 can define a bore or channel 1506 through which the shaft 302 extends. The leaf spring 1502 can have a curved neutral profile, as shown. When in the neutral configuration, an inner surface 1508 of the leaf spring 1502 within the bore or channel 1506 can press against the shaft 302 in locking fashion. The leaf spring 1502 can be configured so that when it is pressed into a straight or less curved configuration, the inner surface 1508 disengages from the shaft 302. It should be appreciated that the leaf spring 1502 and receptacle can have other geometries for locking against and disengaging from the shaft 302. For example, the leaf spring 1502 and receptacle 1504 can employ an angled or featherboard-like locking configuration with respect to the shaft 302.

Referring now to FIG. 16, the proximal wedge 304 can define a slot 390 that extends along the longitudinal direction L and is configured to provide the proximal wedge with compliancy along the transverse direction T. The slot 390 can be configured, for example, to bias side portions 392 of the proximal wedge 304 on opposite sides of the slot 390 towards each other and against the shaft 302 so as to inhibit shaft rotation. Alternatively, the slot 390 can be configured to bias the side portions away from each other and against one or both of the plates 100, 200, for example. The slotted proximal wedge 304 of the present embodiment can be configured such that a tool, e.g., and insertion tool, can engage receptacles 394 of the wedge and thereby counteract the bias force, thereby disengaging the locking connection between the slotted wedge 304 and the shaft 302.

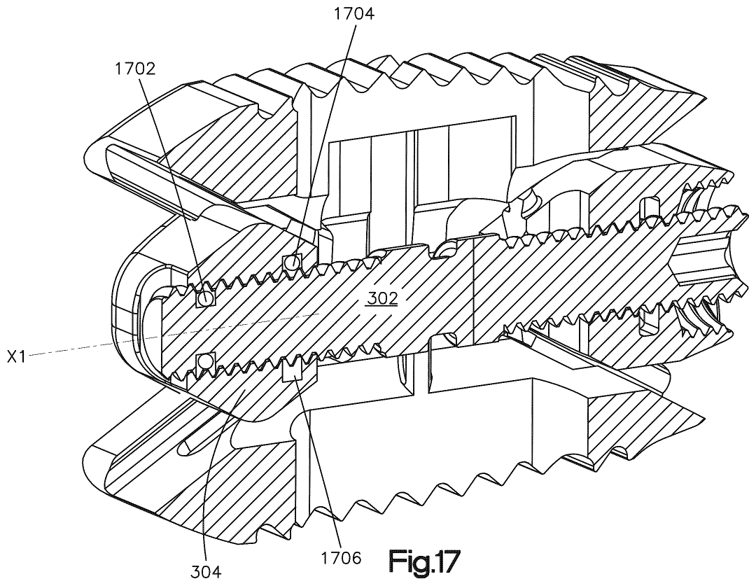

Referring now to FIG. 17, in further embodiments the at least one locking member can comprise a ring member 1702, such as a snap ring that extends about the central axis X1 and around at least a portion of the shaft 302. The ring member 1702 can be configured to provide a degree of friction, resistance, or drag between the shaft 302 and the proximal wedge 304 sufficient to rotationally affix the shaft 302 relative to the proximal wedge 304 after the implant has expanded to the desired height H. Additionally or alternatively, a follower 1704, such as ball-type or ring-type follower, can reside in a receptacle 1706 of one of the wedges, such as the proximal wedge 304, and can be configured to travel along the exterior threads 306 in a manner causing friction, resistance, or drag as the wedge 304 travels along the shaft 302.

It should be appreciated that the embodiments of the implants described herein, in addition to affixing relative positions between various components in a manner effectively locking the implants at their desired heights H1, can further enhance structural support between various components

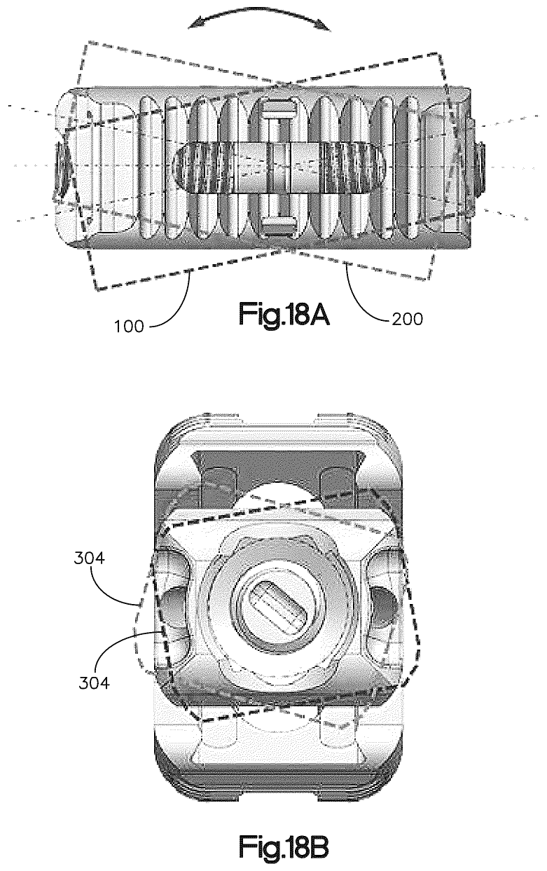

It should be appreciated that the various locking structures described herein for locking the implants at their desired heights H1 (such as by affixing relative positions between various implant components) can provide further benefits and advantages. One such benefit is that such locking structures can further enhance structural support between various components, such as be reducing relative motion between select components. Referring now to FIG. 18A, one such example relative motion that can be advantageously reduced by embodiments herein is angulation between the superior and inferior plates 100, 200 with respect to a horizontal plane L-T or, stated differently, relative angulation about a vertical axis. Referring now to FIG. 18B, another such example relative motion that can be advantageously reduced by embodiments herein is angulation between the proximal and distal wedges 304 about the shaft axis X1. It should be appreciated that the foregoing examples of relative motion are provided as non-limiting examples. Such types of relative motions can cause wear between various components of the implant. Thus, by locking or otherwise affixing relative positions between components of the implant, undesired relative motions can be significantly reduced, which can provide an enhanced healing environment for the patient.

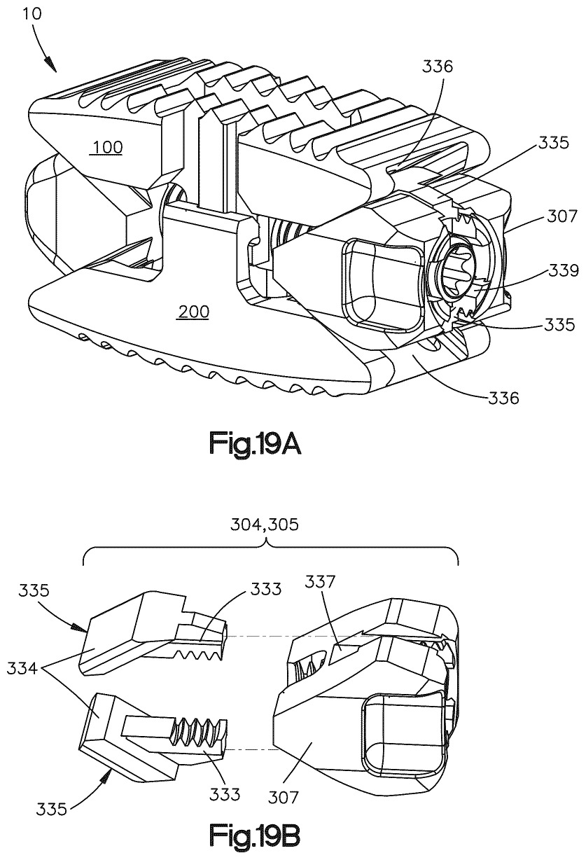

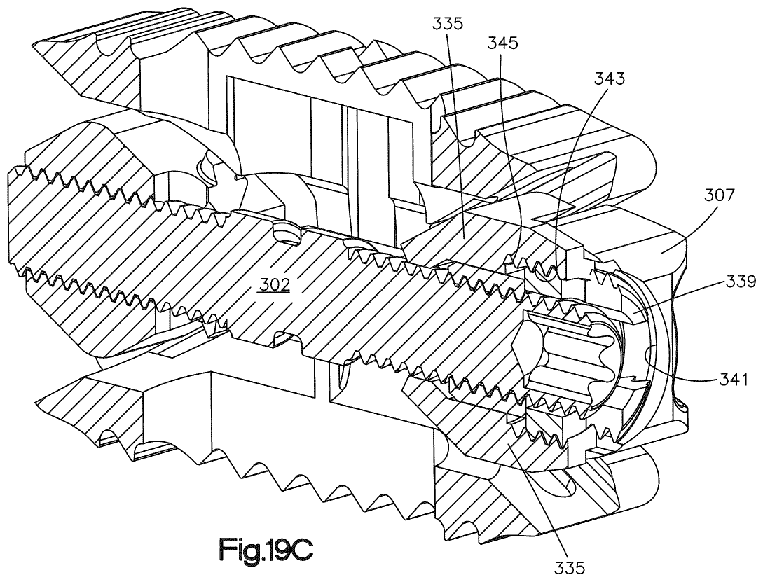

Additional examples of such locking structures and stabilization or reinforcement features will now be described with reference to FIGS. 19A-23D.

Referring now to FIGS. 19A-19C, an implant 10 can have one or more locking structures that are configured to lock at least one wedge 304 to one or both of the endplates 100, 200. The implant 10 can be configured generally similar to the implants 10 described above. In this embodiment, however, the proximal wedge 304 can be a wedge assembly 305 that includes a main wedge body 307 and one or more locking guide members 335, such as a pair of locking guide members 335 vertically spaced from each other. The locking guide members 335 can have respective guide extensions 333 that are configured to extend within, and translate along, respective guide slots 337 of the main wedge body 307 that intersect the superior and inferior push surfaces 330 thereof.

The locking guide members 335 can also define guide formations 334 that are configured to ride within and along the complimentary guide channels 336 of the superior and inferior plates 100, 200. The guide formations 334 of the present embodiment are configured to interlock with the plates 100, 200. For example, the guide formations 334 can have dovetail geometries that are complimentary with those of the guide channels 336, similar to the manner described above with reference to FIGS. 2B-2C. The interlocking geometries in the present embodiment are configured such that proximal translation of the locking guide members 335 relative to the main wedge body 305 effectively clamps the plates 100, 200 to the main wedge body 307.

The proximal wedge assembly 305 includes a locking actuator 339, such as a lock nut 339, which can be configured to reside in a locking receptacle 341 of the main wedge body 307. The lock nut 339 can be disposed along the shaft 302 and can define external threading 345 configured to threadedly engage internal threads 343 of the guide extensions 333. In this manner, rotation of the lock nut 339 about a first rotational direction can translate the locking guide members 335 proximally a distance relative to the main wedge body 307, thereby clamping the plates 100, 200 against the main wedge body 307, effectively locking the implant 10 at the desired height H.

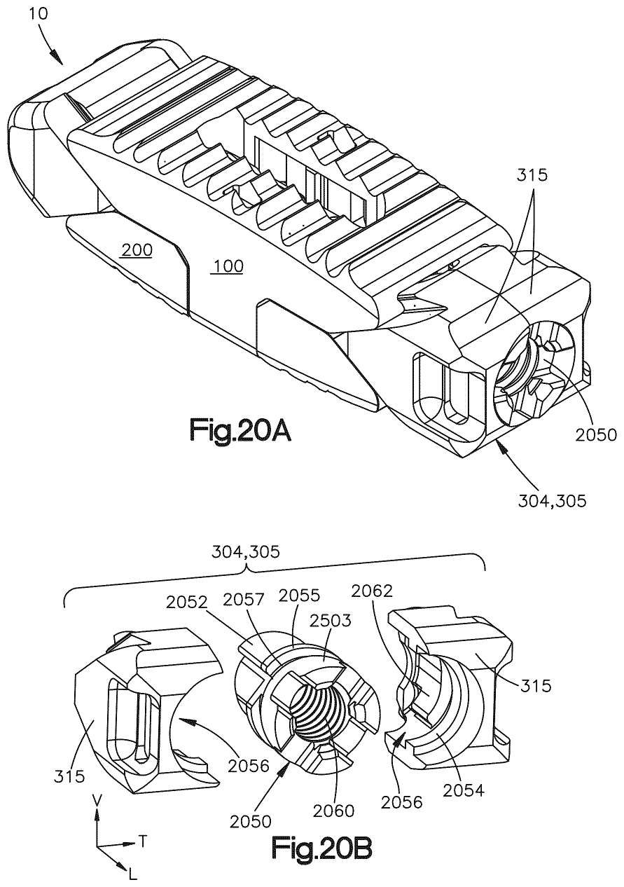

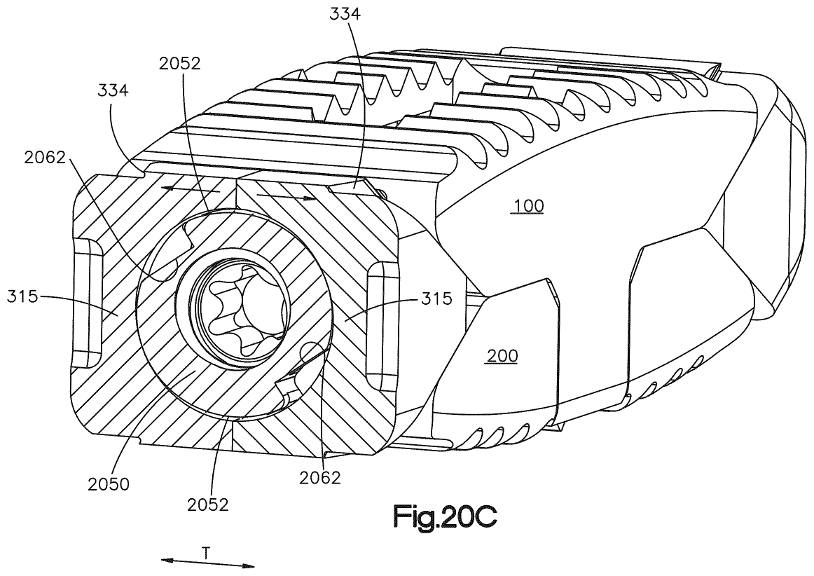

Referring now to FIGS. 20A-20C, another embodiment of an implant 10 is shown that has one or more locking structures that are configured to lock at least one wedge 304 to one or both of the endplates 100, 200. The implant 10 can be configured generally similar to the implants 10 described above. In this embodiment, the proximal wedge 304 can be a wedge assembly 305 that includes a pair of side wedge pieces 315 opposite each other along the transverse direction T. The wedge assembly 305 can include a lock nut 2050 having one or more cam surfaces 2052. Thus, the lock nut 2050 can be referred to as a “cammed lock nut” 2050.

The lock nut 2050 preferably defines interior threads 2060 that are configured to threadedly engage the exterior threads 306 of the proximal threaded region 312 of the shaft 302. The lock nut 2050 of the present embodiment is configured to carry the side wedge pieces 315 along the shaft 302 to expand the implant 10 while the lock nut 2050 remains in a neutral or unlocked position. For example, the lock nut 2050 can define a pair of flanges 2053, 2055 and an annular receptacle 2057 located longitudinally between the flanges 2053, 2055. The annular receptacle 2057 can be configured to engage one or more complimentary follower protrusions 2054 defined within respective receptacles 2056 of the side wedge pieces 315. In this manner, the follower protrusions 2054 can be longitudinally retained within the annular receptacle 2057 between flanges 2053, 2055, which can translate the side wedge pieces 315 along the shaft 302 as the shaft rotates.

After the implant 10 has expanded to the desired height H, the cammed lock nut 2050 can be rotated relative to the side wedge pieces 315 into a locked position. As shown, the cam surfaces 2052 of the lock nut 2050 can be configured to engage cam follower surfaces 2062 defined within the receptacles 2056 of the side wedge pieces 315 in a manner driving the side wedge pieces 315 away from each other along the transverse direction T. As the side wedge pieces 315 separate transversely in this manner, guide formations 334 of the wedge pieces 315 can clamp outwardly against the plates 100, 200, increasing the interlocking fit between the guide formations 334 and the complimentary guide channels 336 sufficient to lock the plates 100, 200 to the wedge assembly 305. With the side wedge pieces 315 transversely separated in this manner, friction between the cam surfaces 2052 and the engaged cam follower surfaces 2062 is configured to prevent the lock nut 2050 from rotating backwards out the locked position.

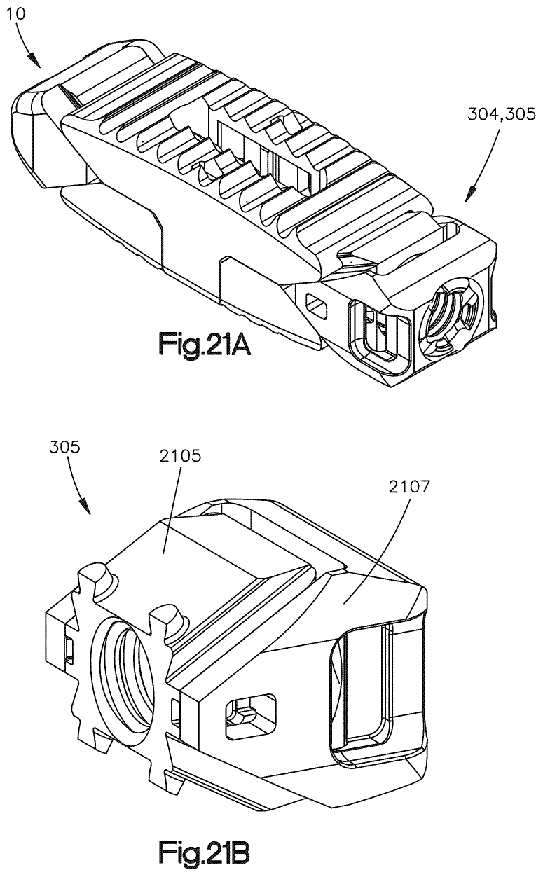

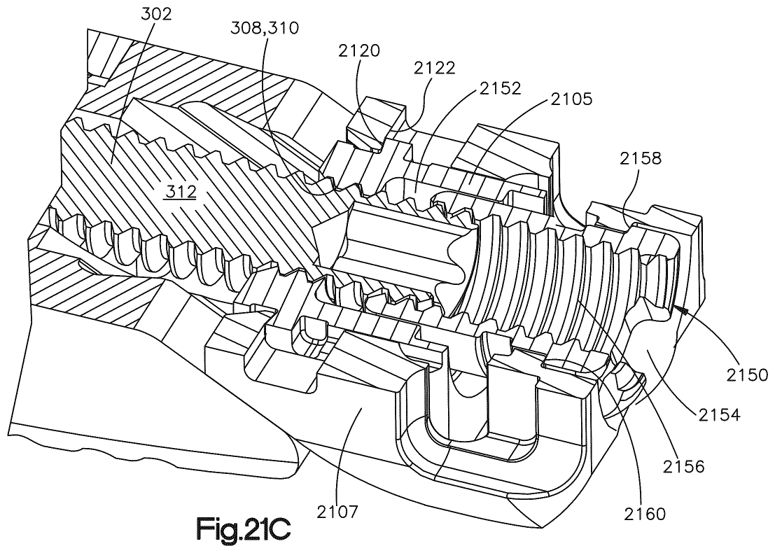

Referring now to FIGS. 21A-21C, another embodiment of an implant 10 is shown that has one or more locking structures that are configured to lock at least one wedge 304 to one or both of the endplates 100, 200. The implant 10 can be configured generally similar to the implants 10 described above. In this embodiment, the proximal wedge 304 can be a wedge assembly 305 that includes a first or inner wedge 2105 and a second or outer wedge 2107 that are coupled together. The inner and outer wedges 2105, 2107 can be configured to translate together along the proximal threaded region 312 of the shaft 302 in an unlocked configuration to facilitate expansion of the implant 10. The inner and outer wedges 2105, 2107 can be further configured to facilitate transition into a locked configuration with the plates 100, 200. For example, a lock actuator, such as a lock nut 2150 that resides within respective locking receptacles 2152, 2154 of the inner and outer wedges 2105, 2107, can be rotated to cause distal translation of the outer wedge 2107 relative to the inner wedge 2105 a distance sufficient to remove any clearance between the outer wedge 2107 and the plates 100, 200. The lock nut 2150 can define interior threads 2156 that threadedly engage the proximal threaded region 312 of the shaft 302 to actuate locking of the outer wedge 2107.

As best shown in FIG. 21C, the inner wedge 2105 can define a bore 308 having interior threads 310 that are threadedly engage with the proximal threaded region 312 of the shaft 302, similar to the manner described above with respect to other embodiments herein. The inner wedge 2105 can define one or more push features, such as opposed transverse protrusions 2120 that engage complimentary push surfaces 2122 defined by the outer wedge 2107. During expansion, the transverse protrusions 2120 of the inner wedge 2105 push distally against the push surfaces 2122 of the outer wedge 2107 so as to advance the inner and outer wedges 2105, 2107 distally together along the proximal threaded region 312 of the shaft 302. When the implant 10 has expanded to the desired height H, the lock nut 2150 can be rotated by a tool to advance the lock nut 2150 distally along the proximal threaded region 312 of the shaft 302 with respect to the inner wedge 2105. The lock nut 2150 has push features, such as push surfaces 2158 that engage complimentary push surfaces 2160 of the outer wedge 2107 so that complimentary push surfaces 2158, 2160 advance the outer wedge 2107 together with the lock nut 2150 relative to the inner wedge 2105, thereby causing the outer wedge 2107 to clamp distally against the plates 100, 200 in locking fashion.

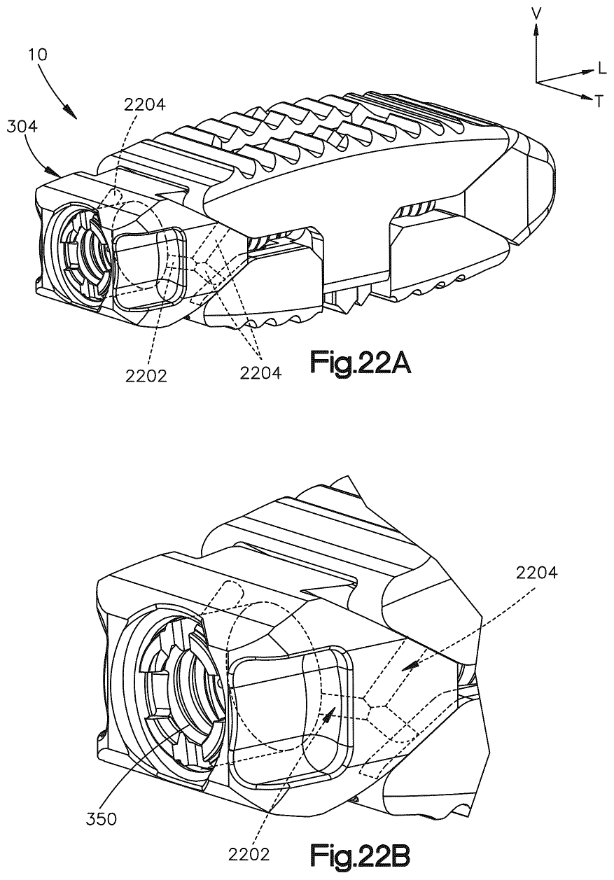

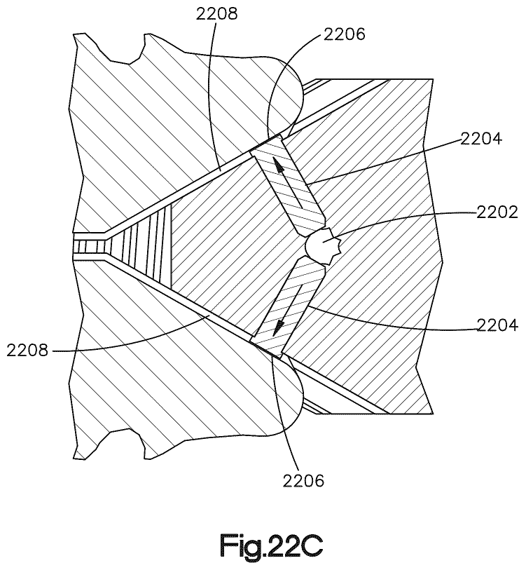

Referring now to FIGS. 22A-22D, in another embodiment, the implant 10 one or more locking structures that include push pins 2202 that drive locking pins 2204 into locking engagement with the plates 100, 200. For example, the proximal wedge 304 can house a pair of push pins 2202 that reside in a neutral or unlocked position during implant 10 expansion. The push pins 2202 can extend along a horizontal plane L-T and can be angled outwards at oblique angles with respect to axis X1. When the implant 10 has expanded to the desired height H1, the push pins 2202 can be actuated, e.g., driven distally so as to impinge against the locking pins 2204, such as by advancing a lock nut 350 within a locking receptacle 352 of the proximal wedge 304. The lock nut 350 and locking receptacle 352 can be configured similarly to those described above with reference to FIGS. 2A-3B. The locking pins 2204 can extend along respective vertical planes (e.g., vertical longitudinal planes V-L). Actuation of the push pins 2202 can drive the locking pins 2204 so that distal ends 2206 thereof abut the plates 100, 200. The distal ends 2206 of the locking pines 2204 can define guide protrusions that extend within complimentary guide slots 2208 recessed within the contact surfaces 332 of the plates 100, 200.

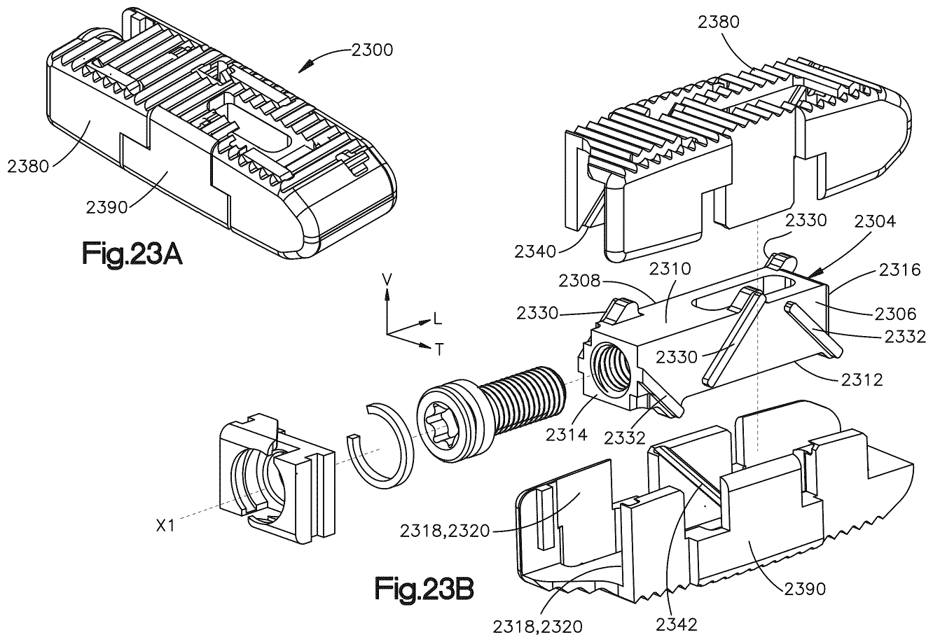

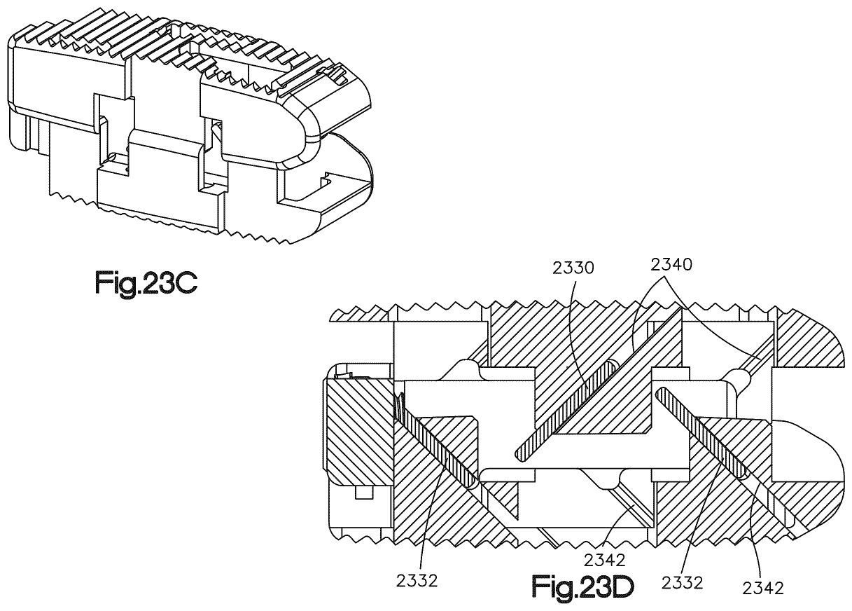

Referring now to FIGS. 23A-23D, in additional embodiments, an expandable implant 2300 can include a single, block-like wedge body 2304 that is actuatable to expand at least one of a superior plate 2380 and an inferior plate 2390 relative to the other along the vertical direction V. As shown, the block-like wedge body 2304 can have substantially rectangular profiles in one or more and up to each of the horizontal L-T plane and both vertical planes V-T, V-L. For example, the wedge body 2304 can define side surfaces 2306, 2308 opposite each other along the transverse direction T, which side surfaces 2306, 2308 are substantially planar along respective vertical-longitudinal planes V-L. The wedge body 2304 can also define superior and inferior surfaces 2310, 2312 opposite each other along the vertical direction V, which superior and inferior surfaces 2310, 2312 are substantially planar along respective horizontal planes L-T. The wedge body 2304 can also define end surfaces 2314, 2316 opposite each other along the longitudinal direction L, which end surfaces 2314, 2316 are substantially planar along respective vertical-transverse planes V-T. Portions of the planar side surfaces 2306, 2308 are configured to slide along complimentary planar surfaces 2318 defined along interior walls 2320 of the plates 2380, 2390. In this manner, the block-like wedge body 2304 significantly increases a contact length between the wedge body 2304 and the superior and inferior plates 2380, 2390.

The wedge body 2304 defines one or more ramp features, such as a plurality of angled rails that extend outwardly from the side surfaces 2306, 2308 along the transverse direction T. The angled rails are configured to ride along and/or within complimentary angled guide channels defined along the interior walls 2320 of the superior and inferior plates 2380, 2390. The angled rails can include a first plurality of rails 2330 that are inclined at a first angle with respect to axis X1 and are configured to ride along an associated first plurality of guide channels 2340 in the superior plate 2380 or inferior plate 2390. The angled rails can also include a second plurality of rails 2332 that are declined at a second angle with respect to axis X1 and are configured to ride along an associated second plurality of guide channels 2342 in the other of the superior plate 2380 and the inferior plate 2390. In this manner, as the wedge body 2304 is actuated longitudinally relative to the plates 2380, 2390, the first plurality of rails 2330 and associated first plurality of guide channels 2340 are configured drive the associated plate 2380, 2390 vertically away from the wedge body 2304, and the second plurality of rails 2332 and associated second plurality of guide channels 2342 are configured drive the other plate 2380, 2390 vertically away from the wedge body 2304 in the opposite vertical direction, thereby expanding the implant 2300.

It should be appreciated that the implant 2300 of the present embodiment, and various aspects thereof, can be configured as more fully described in U.S. Pat. No. 10,799,366, issued Oct. 13, 2020, in the name of Davis et al., the entire disclosure of which is hereby incorporated by reference herein.

It should further be appreciated when a numerical preposition (e.g., “first”, “second”, “third”) is used herein with reference to an element, component, dimension, or a feature thereof, such numerical preposition is used to distinguish said element, component, dimension, and/or feature from another such element, component, dimension and/or feature, and is not to be limited to the specific numerical preposition used in that instance. For example, a “first” wedge, formation, flange, or direction, by way of non-limiting examples, can also be referred to as a “second” wedge, formation, flange, or direction in a different context without departing from the scope of the present disclosure, so long as said elements, components, dimensions and/or features remain properly distinguished in the context in which the numerical prepositions are used.

Although the disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. In particular, one or more of the features from the foregoing embodiments can be employed in other embodiments herein. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. An expandable intervertebral cage, comprising:
a superior plate and an inferior plate opposite each other along a vertical direction;
a distal wedge and a proximal wedge each disposed between the superior and inferior plates, the distal and proximal wedges located opposite each other along a longitudinal direction substantially perpendicular to the vertical direction, the distal and proximal wedges each defining ramped surfaces configured to engage complimentary ramped surfaces of the superior and inferior plates in a manner increasing a distance between the superior and inferior plates along the vertical direction;
an actuator located between the superior and inferior plates, the actuator defining a central axis oriented along the longitudinal direction, the actuator coupled to the distal and proximal wedges such that rotation of the actuator about the central axis moves at least one of the proximal and distal wedges relative to the other of the proximal and distal wedges along the longitudinal direction in a manner increasing the distance; and
at least one locking component insertable within a receptacle at least partially defined by the proximal wedge, wherein the at least one locking component is configured to transition from an unlocked configuration, in which the actuator is rotatable about the central axis relative to the proximal wedge, to a locked configuration, in which the actuator is substantially rotationally affixed relative to the proximal wedge.

2. An expandable intervertebral cage, comprising:
a superior plate and an inferior plate opposite each other along a vertical direction;
a distal wedge and a proximal wedge each disposed between the superior and inferior plates, the distal and proximal wedges located opposite each other along a longitudinal direction substantially perpendicular to the vertical direction, the distal and proximal wedges each defining ramped surfaces configured to engage complimentary ramped surfaces of the superior and inferior plates in a manner increasing a distance between the superior and inferior plates along the vertical direction;
an actuator located between the superior and inferior plates, the actuator defining a central axis oriented along the longitudinal direction, the actuator coupled to the distal and proximal wedges such that rotation of the actuator about the central axis moves at least one of the proximal and distal wedges relative to the other of the proximal and distal wedges along the longitudinal direction in a manner increasing the distance; and
at least one locking component carried by the proximal wedge, wherein the at least one locking component is configured to transition from an unlocked configuration, in which the proximal wedge is translatable relative to at least one of the superior and inferior plates along the longitudinal direction, to a locked configuration, in which the proximal wedge is substantially longitudinally affixed relative to the at least one of the superior and inferior plates.

3. An expandable intervertebral cage elongate along a longitudinal direction, comprising:
a superior plate and an inferior plate opposite each other along a vertical direction;
a single wedge body having first and second side surfaces opposite each other along a transverse direction that is substantially perpendicular to the vertical direction, wherein the first and second side surfaces are substantially planar from a proximal end of the wedge body to a distal end of the wedge body, and wherein the first and second side surfaces are configured to interface with respective planar surfaces defined along respective interior walls of the superior plate and the inferior plate;
the single wedge body defining a plurality of angled rails extending outwardly from the first and second side surfaces along the transverse direction, wherein the angled rails are oriented at respective oblique angles with respect to a central axis of the cage that is oriented along the longitudinal direction, wherein the plurality of angled rails are configured to ride along a respective plurality of angled guide channels defined along the interior walls of the superior plate and the inferior plate in a manner increasing a distance between the superior and inferior plates along the vertical direction;
an actuator located between the superior and inferior plates and extending along the central axis, the actuator coupled to the wedge body such that rotation of the actuator about the central axis moves the wedge body along the longitudinal direction in a manner increasing the distance.

4. An expandable intervertebral cage, comprising:
a superior plate and an inferior plate opposite each other along a vertical direction;
a distal wedge and a proximal wedge each disposed between the superior and inferior plates, the distal and proximal wedges located opposite each other along a longitudinal direction substantially perpendicular to the vertical direction, the distal and proximal wedges each defining ramped surfaces configured to engage complimentary ramped surfaces of the superior and inferior plates in a manner increasing a distance between the superior and inferior plates along the vertical direction;
an actuator located between the superior and inferior plates, the actuator defining a central axis oriented along the longitudinal direction, the actuator coupled to the distal and proximal wedges such that rotation of the actuator about the central axis moves at least one of the proximal and distal wedges relative to the other of the proximal and distal wedges along the longitudinal direction in a manner increasing the distance; and
a locking mechanism configured to maintain the increased distance, wherein the locking mechanism comprises a ratchet assembly that includes:
a first plurality of ratchet teeth defined along a flexible ratchet support; and
a second plurality of ratchet teeth, wherein at least one tooth of the first plurality of ratchet teeth is configured to travel against the second plurality of ratchet teeth in sequential interdigitating fashion in a first movement direction as the distance increases, and the second plurality of ratchet teeth are configured to prevent movement of the at least one tooth in a second movement direction opposite the first movement direction.
